(19) United States
(12) Patent Application Publication
(10) Pub. No.: US 2024/0216347 A1
(43) Pub. Date: Jul. 4, 2024

(54) BEZUCLASTINIB FORMULATIONS

(71) Applicant: Cogent Biosciences, Inc., Waltham, MA (US)

(72) Inventors: Bradley Barnett, Lafayette, CO (US); Elodie Rosinovsky, Maynard, MA (US); Hulya Sahin, Piscataway, NJ (US); Michael M. Crowley, Austin, TX (US)

(21) Appl. No.: 18/393,543

(22) Filed: Dec. 21, 2023

Related U.S. Application Data
(60) Provisional application No. 63/476,812, filed on Dec. 22, 2022.

(51) Int. Cl. A61K 31/437; A61K 9/20; A61K 31/404

(52) U.S. Cl. CPC A61K 31/437; A61K 9/2054; A61K 31/404

(57) ABSTRACT

The disclosure relates to formulations of bezuclastinib, methods of making such formulations, and methods of using such formulations in the treatment of conditions, diseases, or disorders that would benefit from modulation of c-Kit protein kinases and/or mutant c-kit protein kinases.

### CROSS-REFERENCE TO RELATED APPLICATIONS

This application claims priority to U.S. provisional patent application No. 63/476,812 filed on Dec. 22, 2022, the contents of which are incorporated by reference herein in its entirety.

### TECHNICAL FIELD

The disclosure relates to novel dosage forms comprising 3,4-dimethyl-N-(2-phenyl-1H-pyrrolo[2,3-b]pyridin-5-yl)-1H-pyrazole-5-carboxamide. The novel dosage forms exhibit improved properties, including allowing increased dosages.

### BACKGROUND

Receptor protein tyrosine kinases (RPTKs) regulate key signal transduction cascades that control cellular growth and proliferation. The Stem Cell Factor (SCF) receptor C-Kit is a type III transmembrane RPTK that includes five extracellular immunoglobulin (IG) domains, a single transmembrane domain, and a split cytoplasmic kinase domain separated by a kinase insert segment. C-Kit (also known as KIT, CD117, and stem cell factor receptor) plays an important role in the development of melanocytes, mast, germ, and hematopoietic cells.

Aberrant expression and/or activation of c-Kit and/or a mutant form(s) of c-Kit has been implicated in a variety of pathologic states (Roskoski, 2005, Biochemical and Biophysical Research Comm. 338:1307-1315). For example, evidence for a contribution of c-Kit to neoplastic pathology includes its association with leukemias and mast cell tumors, small cell lung cancer, testicular cancer, and some cancers of the gastrointestinal tract and central nervous system. In addition, c-Kit has been implicated in playing a role in carcinogenesis of the female genital tract sarcomas of neuroectodermal origin, and Schwann cell neoplasia associated with neurofibromatosis. It was found that mast cells are involved in modifying the tumor microenvironment and enhancing tumor growth (Yang et al., J Clin Invest. 2003, 112:1851-1861; Viskochil, J Clin Invest. 2003, 112:1791-1793).

Therefore, pharmaceutical formulations comprising a c-Kit inhibitor would be of high therapeutic value in the treatment of patients suffering from disease or condition, such as Acute Myeloid Leukemia (AML), Gastrointestinal Stromal Tumors (GIST), Mast Cell Leukemia (MCL) and mastocytosis. There is a dire need for oral formulations of c-Kit inhibitors, as oral drug delivery is a popular route of administration because of its versality, ease of administration and patient compliance.

### BRIEF SUMMARY

The following aspects and embodiments thereof described below are meant to be exemplary and illustrative, not limiting in scope.

In one aspect, the disclosure relates to a pharmaceutical formulation comprising 3,4-dimethyl-N-(2-phenyl-1H-pyrrolo[2,3-b]pyridin-5-yl)-1H-pyrazole-5-carboxamide (Compound (I)), and a pharmaceutically acceptable polymer. In one aspect, Compound (I) is dispersed in a polymer matrix formed from the pharmaceutically acceptable polymer.

In one aspect of the disclosure, the pharmaceutical formulation comprises a spray-dried solid dispersion.

In one aspect of the disclosure, Compound (I) and the pharmaceutically acceptable polymer are in a spray-dried solid dispersion.

In one aspect of the disclosure, Compound (I) is in an amorphous form.

In one aspect of the disclosure, the Compound (I) is in a free base form.

In one aspect of the disclosure, the pharmaceutically acceptable polymer is hydroxypropyl methyl cellulose acetate succinate (HPMCAS).

In one aspect of the disclosure, the pharmaceutically acceptable polymer is hydroxypropyl methyl cellulose acetate succinate grade H (HPMCAS-H).

In aspects of the disclosure, the spray-dried solid dispersion comprises at least about 1% by weight to at least about 25% by weight of Compound (I).

The spray-dried solid dispersion of the disclosure comprises at least about 75% by weight to at least about 99% by weight of the pharmaceutically acceptable polymer.

The weight ratio of Compound (I) to the pharmaceutically acceptable polymer is from about 1:3 to about 1:99 in the spray-dried solid dispersion of the disclosure.

The spray-dried solid dispersion of the disclosure can further comprise a solvent. The solvent is a combination of water and tetrahydrofuran. The volume ratio of water to tetrahydrofuran is from about 1:2 to about 1:99.

The disclosure also relates to a tablet that comprises a spray-dried solid dispersion comprising Compound (I), a pharmaceutically acceptable polymer, wherein Compound (I) is dispersed in a polymer matrix formed from the pharmaceutically acceptable polymer, one or more pharmaceutically acceptable ingredients selected from the group consisting of one or more binders, one or more buffering agents, one or more diluents, one or more disintegrants, one or more fillers, one or more film coatings, one or more lubricants, one or more glidants, and one or more surfactants.

A tablet according to the disclosure comprises one or more pharmaceutically acceptable ingredients, such as, but not limited to colloidal silicon dioxide, croscarmellose sodium, sodium stearyl fumarate, mannitol, and microcrystalline cellulose.

The disclosure also provides a tablet comprising Compound (I) dispersed in a polymer matrix formed from a pharmaceutically acceptable polymer, and one or more pharmaceutically acceptable ingredients selected from the group consisting of one or more binders, one or more buffering agents, one or more diluents, one or more disintegrants, one or more fillers, one or more film coatings, one or more lubricants, one or more glidants, and one or more surfactants.

In the tablets of the disclosure, Compound (I) that is dispersed in a polymer matrix formed from a pharmaceutically acceptable polymer is a spray-dried solid dispersion.

In the tablets of the disclosure, the Compound (I) is in an amorphous and free base form and the pharmaceutically acceptable polymer is hydroxypropyl methyl cellulose acetate succinate (HPMCAS).

In the tablets of the disclosure, the Compound (I) is in an amorphous and free base form and the pharmaceutically acceptable polymer is hydroxypropyl methyl cellulose acetate succinate grade H (HPMCAS-H).

A tablet of the disclosure comprises at least about 1% by weight to at least about 20% by weight of Compound (I).

A tablet of the disclosure also comprises at least about 10% by weight to at least about 90% by weight of the pharmaceutically acceptable polymer.

In aspects of the disclosure, the tablet comprises at least about 3% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In specific aspects, a tablet of the disclosure comprises at least about 1% by weight to at least about 20% by weight of Compound (I), at least about 10% by weight to at least about 90% by weight of hydroxypropyl methyl cellulose acetate succinate grade H (HPMCAS-H), and at least about 3% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

The disclosure also relates to a method of treating a subject suffering from disease or condition comprising administering orally to the subject a tablet as disclosed herein.

In some aspects, the method further includes administering a therapeutic agent other than Compound (I) in combination with the tablet. In aspects of the disclosure, the therapeutic agent other than Compound (I) is a receptor tyrosine kinase (RTK) inhibitor, such as sunitinib malate.

In aspects of the disclosure, the disease or condition treated by oral administration of the tablet disclosed herein includes, but not limited to Acute Myeloid Leukemia (AML), Gastrointestinal Stromal Tumors (GIST), and mastocytosis.

In aspects of the disclosure, the mastocytosis that is treated by oral administration of the tablet disclosed herein is Advanced Systemic Mastocytosis (AdvSM), Nonadvanced Systemic Mastocytosis (NonAdvSM), Indolent Systemic Mastocytosis (ISM) and Smoldering Systemic Mastocytosis (SSM).

In other aspects of the disclosure, the tablet disclosed herein is taken once daily, twice daily, or is taken continuously in 28-day cycles.

In another aspect, the methods of the disclosure result in a target area-under-the-curve (AUC) from 500 to 80,000 (ng·h/mL) in 500 ng·h/mL increments following a single oral dose in the subject.

In yet another aspect, the methods of the disclosure result in a maximal plasma concentration (Cₘₐₓ) from 100 to 800 (ng/mL) in 100 ng/ml increments following a single oral dose in the subject.

In aspects of the methods of the disclosure, the once daily, steady state target area-under-the-curve (AUC) is from 30,000 to 50,000 (ng·h/mL) in 500 ng·h/mL increments when 600 mg dose of bezuclastinib are co-administered with 37.5 mg of sunitinib malate.

In other aspects of the methods of the disclosure, the once daily, steady state maximal plasma concentration (Cₘₐₓ) is from 1,500 to 2,500 (ng/mL) in 500 ng·h/mL increments when 600 mg dose of bezuclastinib are co-administered with 37.5 mg of sunitinib malate.

### BRIEF DESCRIPTION OF THE DRAWINGS

FIG. 1 is a chart showing polymer screening with API using the KinetiSol® technology.

FIG. 2 is a microcentrifuge assay showing the non-sink total drug dissolution testing results of SDD samples compared to crystalline API in simulated intestinal fluid with 0.5% bile salt concentration at a pH of 6.5 and 37° C.

FIG. 3 is a ultracentrifuge assay showing the non-sink free drug dissolution testing results of SDD samples compared to crystalline API in simulated intestinal fluid with 0.5% bile salt concentration at a pH of 6.5 and 37° C.

FIG. 4 is plot showing the non-sink, total drug dissolution testing results of 10%, 15% and 20% drug loaded SDD samples with a gastric transfer at 37° C.

FIG. 5 is a plot showing the non-sink, free drug dissolution testing results of 10%, 15% and 20% drug loaded SDD samples with a gastric transfer at 37° C.

FIG. 6 is a process flow diagram showing the steps towards preparation of Formulations A and B.

FIG. 7 shows the plasma concentration time curves of the tablets made with SDD and KSD amorphous dispersions in non-human primates.

FIG. 8 shows the area under the curve of the tablets made with SDD and KSD amorphous dispersions in non-human primates.

FIG. 9 shows the plasma concentration time curves of Formulations A and B at different doses through 336 hours.

FIG. 10 shows the plasma concentration time curves of Formulations A and B at different doses through 24 hours.

FIG. 11 compares the Cₘₐₓ of Formulations A & B at different doses.

FIG. 12 compares the Area Under the Curve (AUC) of Formulations A & B at different doses.

FIG. 13 compares the Geometric Mean and 90% Confidence Intervals of the Cₘₐₓ and Area Under the Curve of Formulations A and B at different doses.

### DETAILED DESCRIPTION

The present disclosure relates to spray-dried solid dispersions and tablets comprising 3,4-dimethyl-N-(2-phenyl-1H-pyrrolo[2,3-b]pyridin-5-yl)-1H-pyrazole-5-carboxamide (Compound I). The chemical structure of Compound (I) is shown below.

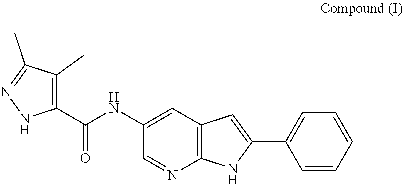 [SMILES: Cc1n[nH]c(C(=O)Nc2cnc3[nH]c(-c4ccccc4)cc3c2)c1C]

Compound (I) is also referred to by its International Nonproprietary Name Bezuclastinib.

Compound (I) described herein is kinase modulator that is active on c-Kit protein kinases or mutant c-Kit protein kinases. The synthesis of Compound (I) and methods of treating diseases and conditions associated with aberrant activity of the c-Kit protein kinases and/or mutant c-Kit protein kinases have previously been disclosed in U.S. Pat. No. 9,676,748 B2 and U.S. Pat. No. 10,301,280 B2, the entire contents of which are incorporated by reference herein.

Compound (I) was first disclosed in U.S. Pat. No. 9,676,748 B2 and U.S. Pat. No. 10,301,280 B2. In the almost 10 years since, Compound (I) has proven to be difficult to prepare in a tablet form suitable for commercial development. In particular, Compound (I) has a very high melting point (>365° C.) and is practically insoluble in aqueous conditions (0.32 ug/mL at pH 7.0), and most organic solvents. In addition, no suitable salt or co-crystal has been found for of Compound (I), which itself readily crystalizes when forming tablets. Finally, existing tablet forms of Compound (I) allowed a relatively small proportion of API to be loaded into the tablet form, necessitating repeated dosing of many tablets, potentially jeopardizing patient compliance.

Applicant has surprisingly discovered a process to address the low solubility of Compound (I) and to formulate a tablet comprising Compound (I). In this process, Compound (I) is in an amorphous and free base form. The process is a elegant, rapid, cost effective, time-saving and industrially convenient.

The low solubility problem of Compound (I) was overcome by preparing spray dried dispersions (SDDs). Specifically, an amorphous molecular dispersal of Compound (I) in a polymer matrix, such as hydroxypropyl methyl cellulose acetate succinate grade H (HPMCAS-H) was created by dissolving Compound (I) and the polymer in a solvent system, such as THF and water and then spray-drying the solution. Such a formulation surprisingly allowed a greater amount of Compound (I) to be loaded into the tablet than previous formulations, and the formulation also delivered more of Compound (I) to the blood of human subjects than previous formulations.

The disclosure thus relates to a tablet comprising spray dried dispersion of Compound (I) having desired dissolution profile and desired stability.

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

As used herein, the term “active agent,” “active pharmaceutical ingredient,” or “API” refers to a pharmaceutically active agent or a drug. Furthermore, these terms can also refer to “Compound (I)” or “bezuclastinib.” All these terms also may be used interchangeably.

As used herein, the term “amorphous” refers to a solid form of Compound (I) that is not crystalline. An amorphous solid does not display a definitive X-ray diffraction pattern with sharp maxima; it is a thermodynamically non-equilibrium material that exhibits no long-range periodicity.

As used herein, the terms “approximately” and “about” refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “combination therapy” is a treatment that includes the administration of two or more therapeutic agents, e.g., Compound (I) and a receptor tyrosine kinase (RTK) inhibitor, such as but not limited to sunitinib malate, to a patient. The two or more therapeutic agents may be delivered at the same time, e.g., in separate pharmaceutical compositions or in the same pharmaceutical composition, or they may be delivered at different times. For example, they may be delivered concurrently or during overlapping time periods, and/or one therapeutic agent may be delivered before or after the other therapeutic agent(s). Treatment with a combination therapy optionally includes treatment with either single agent, preceded or followed by a period of concurrent treatment with both agents. However, it is contemplated that during some time period, effective amounts of the two or more therapeutic agents are present within the patient.

As used herein, the term “Compound (I)” refers to 3,4-dimethyl-N-(2-phenyl-1H-pyrrolo [2,3-b]pyridin-5-yl)-1H-pyrazole-5-carboxamide.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, the term “excipient” herein includes any substance used as a vehicle for delivery of the active ingredient to a subject, and any substance added to the active ingredient, for example to improve its handling properties or to permit the resulting composition to be formed into an orally deliverable unit dose having the desired shape and consistency. Excipients can include, by way of illustration and not by limitation, a filler, a binder, a surfactant, a disintegrant, a glidant, a lubricant or a combination thereof, substances added to improve appearance of a dosage form, and any other substance other than the active ingredient conventionally used in the preparation of oral dosage forms. The term “excipient” includes inert substances as well as functional excipients that may result in beneficial properties of the composition. Exemplary excipients include but are not limited to polymers, glidants, sugars, lubricants, salts, buffers, fats, fillers, disintegrating agents, binders, surfactants, high surface area substrates, flavorants, carriers, matrix materials, and so forth.

As used herein, the term “in vitro” refers to events that occur in an artificial environment, e.g., in a test tube or reaction vessel, in cell culture, in a petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “mammal” includes humans and both domestic animals such as laboratory animals and household pets (e.g., cats, dogs, swine, cattle, sheep, goats, horses, rabbits), and non-domestic animals such as wildlife and the like.

As used herein, the term “pharmaceutically acceptable” refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

As used herein, the term “pharmaceutical composition” refers to the composition comprising the API along with pharmaceutically acceptable excipients for the oral delivery of the API to mammals.

As used herein, the term “spray-dried solid dispersion,” or “spray-dried dispersion” (SDD) refers to a dispersion comprising a drug and a polymer, wherein the drug is non-crystalline and is amorphous. An amorphous dispersion of the drug can be prepared by various manufacturing processes such as spray drying, co-precipitation, or hot melt extrusion. In embodiments of this disclosure, spray-drying procedures are used. A spray-dried dispersion (SDD) is a single-phase, amorphous molecular dispersion of a drug in a polymer matrix; it is an amorphous solid in which the drug is molecularly “dissolved” in a solid matrix. A spray-dried dispersion can be made by dissolving the drug and a polymer in an organic solvent to produce a solution, followed by spray-drying the solution. Techniques for preparing solid dispersions of an amorphous drug in a polymer are disclosed in, for example, U.S. Pat. Nos. 9,095,585 and 9,468,604, the contents of each of which are hereby incorporated by reference in their entirety herein. Solid dispersions are also described in, for example, U.S. Pat. No. 8,263,128.

As used herein, the term “subject,” “individual,” or “patient” are used interchangeably and include any animal to which a composition in accordance with the disclosure may be administered. e.g., for experimental, diagnostic, prophylactic, and/or therapeutic purposes. Typical subjects include animals, such as, but not limited to mammals, such as, but not limited to mice, rats, rabbits, non-human primates, and humans.

As used herein, the term “stable” means amorphous form of a Compound (I) that does not convert to any other solid form and contains less than 5% (wt/wt) total other forms (or e.g., less than 4% w/w, less than 3% w/w, less than 2% w/w) when stored at a temperature of up to about 40° C., and at a relative humidity of about 25% to about 75% for at least about three months.

As used herein, the term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.”

As used herein, the term “effective amount” or “therapeutically effective amount” refers to the amount of the composition which, when administered to a mammal, preferably a human, is sufficient to effect treatment in the mammal, preferably a human. The amount of composition which constitutes a “therapeutically effective amount” will vary depending on the condition and its severity, the manner of administration, and the age of the mammal to be treated, but can be determined routinely by one of ordinary skill in the art having regard to his own knowledge and to this disclosure.

As used herein, the term “oral formulation,” refers to a composition or medium used to administer a compound as disclosed herein (e.g., Compound (I)) to a subject in need thereof by oral administration. Typically, an oral formulation is administered via the mouth, however, “oral formulation” as used herein is intended to cover any substance which is administered to a subject and is absorbed across a membrane, e.g., a mucosal membrane, of the gastrointestinal tract, including, e.g., the mouth, esophagus, stomach, small intestine, large intestine, and colon. In some embodiments, the oral formulation is a pharmaceutical composition. In some embodiments, the oral formulation is a pharmaceutical composition administered to a subject in need thereof via the mouth.

As used herein, the term “solid dispersion” means any solid composition having at least two components. In certain embodiments, a solid dispersion as disclosed herein includes an active ingredient (e.g. Compound (I)) dispersed among at least one other component, for example a polymer (e.g. HPMCAS-H).

As used herein, the term “treating” or “treatment” as used herein covers the treatment of the disease or condition of interest in a mammal, preferably a human, having the disease or condition of interest, and includes preventing the disease or condition from occurring in a mammal, in particular, when such mammal is predisposed to the condition but has not yet been diagnosed as having it, inhibiting the disease or condition, i.e., arresting its development, relieving the disease or condition, i.e., causing regression of the disease or condition, or relieving the symptoms resulting from the disease or condition, i.e., relieving pain without addressing the underlying disease or condition. The term “treating” also includes any effect, e.g., lessening, reducing, modulating, or eliminating, that results in the improvement of the condition, disease, disorder and the like.

In general, a solid-state form, such as a crystal form or amorphous form, may be characterized by modulated differential scanning calorimetry (mDSC), powder X-ray diffraction (PXRD), near infrared spectroscopy (NIR), or any other standard analytical technique. For example, mDSC assesses the thermal properties of an SDD; for an amorphous SDD, analysis by mDSC will yield a single glass transition temperature. mDSC can also detect crystalline phase separation, as the crystalline phase will show a unique thermal signal. PXRD uses x-rays to identify crystal form in solid powders and can be used to analyze SDDs, for example to confirm an SDD is a single amorphous phase, with no measurable crystalline material. As is well-known in the art, the graphical data potentially provides additional technical information to further define the respective solid-state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. A crystal is composed of atoms periodically arranged in a 3D space while in amorphous materials atoms are randomly distributed in the 3D space. As a result, the X-ray diffractogram of a crystalline material will display narrow peaks of high intensity due the fact that the x-rays are scattered in only certain directions (due to the periodic arrangement of the atoms). In contrast, the X-ray diffractogram of an amorphous material generally displays broad peaks (halo pattern) of low intensity because the x-rays are scattered in many different directions leading to large bumps distributed over a wide range (2 Theta).

The compositions of the present disclosure can comprise, consist essentially of, or consist of, the components disclosed.

All percentages, parts and ratios are based upon the total weight of the compositions and all measurements made are at about 25° C., unless otherwise specified.

All ranges recited herein include the endpoints, including those that recite a range “between” two values. Term “substantially” and “about” is to be construed as modifying a term or value such that it is not an absolute. This includes, at very least, the degree of expected experimental error, technique error and instrument error for a given technique used to measure a value.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual excipients, polymers, compounds, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Spray-Dried Dispersions

Dispersions of the active agent and pharmaceutically acceptable polymer as described herein are made by a spray-drying process. As used herein, the term “spray-dried dispersion” or “spray-dried powdered dispersion” means a product of a spray-drying process wherein the product comprises a dispersion of at least one active agent and at least one excipient, such as a polymer.

In some embodiments, the active agent is 3,4-dimethyl-N-(2-phenyl-1H-pyrrolo[2,3-b]pyridin-5-yl)-1H-pyrazole-5-carboxamide (Compound I). In some embodiments, the polymer is a pharmaceutically acceptable polymer, such as hydroxypropyl methyl cellulose acetate succinate grade H (HPMCAS-H).

In the spray-drying process, the active agent and the polymer are dissolved in a common solvent. “Common” here means that the solvent, which can be a mixture of compounds, will dissolve both the active agent and the polymer. After both active agent and polymer have been dissolved, the solvent is rapidly removed by evaporation in the spray-drying apparatus, resulting in the formation of a substantially homogeneous solid dispersion. In such dispersions, the active agent is dispersed as homogeneously as possible throughout the polymer and can be thought of as a solid solution of active agent dispersed in the polymer. In aspects of the disclosure, the active agent and the polymer are dissolved in a solvent that is a combination of water and tetrahydrofuran.

The solvent is removed by the spray-drying process. The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). Further, additional process and spray-drying techniques and equipment are described generally in U.S. Pat. Nos. 8,343,550 and 7,780,988, the contents of which are incorporated herein by reference in their entirety for all purposes. The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, a portion of the heat required for evaporation of solvent may be provided by heating the spray solution.

The drying gas may be virtually any gas, but to minimize the risk of fire or explosions due to ignition of flammable vapors, and to minimize undesirable oxidation of the drug, concentration-enhancing polymer, or other materials in the dispersion, an inert gas such as nitrogen, nitrogen-enriched air, or argon is utilized. The temperature of the drying gas at the gas inlet of apparatus is typically from about 60° C. to about 300° C. The temperature of the product particles, drying gas, and evaporated solvent at the outlet or distal end of collection cone typically ranges from about 0° C. to about 100° C.

Solvents suitable for spray-drying process can be any organic compound in which the active agent and polymer are mutually soluble. The solvent should have relatively low toxicity and be removed from the dispersion to a level that is acceptable according to The International Committee on Harmonization (ICH) guidelines. Removal of solvent to this level may require a subsequent processing step such as tray-drying or secondary drying. In some embodiments, the solvent comprises tetrahydrofuran (THF). Mixtures of solvent and water are suitable as long as the polymer and API are sufficiently soluble to make the spray-drying process practicable. In some embodiments, the water:solvent mixture is water:THF. In some embodiments, the solvent is 100% THF.

The composition of the solvent-bearing feed will depend on the desired ratio of drug-to-polymer in the dispersion and the solubility of the drug and polymer in the solvent. Generally, it is desirable to use as high a combined drug and polymer concentration in the solvent-bearing feed as possible, provided the drug and polymer are dissolved in the solvent at the temperature range of the process, to reduce the total amount of solvent that must be removed to form the solid amorphous dispersion.

The average residence time of particles in the drying chamber should be at least 10 seconds, preferably at least 20 seconds. Typically, following solidification, the powder formed stays in the spray-drying chamber for about 5 to 60 seconds, causing further evaporation of solvent. The final solvent content of the solid dispersion as it exits the dryer should be low, since this reduces the mobility of drug molecules in the dispersion, thereby improving its stability. Generally, the solvent content of the dispersion as it leaves the spray-drying chamber should be less than about 10 wt %. In some embodiments, the solvent content of the dispersion as it leaves the spray-drying chamber is less than about 9 wt %. In some embodiments, the solvent content of the dispersion as it leaves the spray-drying chamber is less than about 8 wt %. In some embodiments, the solvent content of the dispersion as it leaves the spray-drying chamber is less than about 7 wt %. In some embodiments, the solvent content of the dispersion as it leaves the spray-drying chamber is less than about 6 wt %. In some embodiments, the solvent content of the dispersion as it leaves the spray-drying chamber is less than about 5 wt %. In some embodiments, the solvent content of the dispersion as it leaves the spray-drying chamber is less than about 4 wt %. In some embodiments, the solvent content of the dispersion as it leaves the spray-drying chamber is less than about 3 wt %. In some embodiments, the solvent content of the dispersion as it leaves the spray-drying chamber is less than about 2 wt %. In some embodiments, the solvent content of the dispersion as it leaves the spray-drying chamber is less than about 1 wt %. A subsequent processing step, such as tray-drying, may be used to remove the solvent to this level.

In one aspect, provided herein a spray-dried solid dispersion (SDD) comprising: (a) 3,4-dimethyl-N-(2-phenyl-1H-pyrrolo[2,3-b]pyridin-5-yl)-1H-pyrazole-5-carboxamide (Compound (I)), (b) a pharmaceutically acceptable polymer, and wherein Compound (I) is dispersed in a polymer matrix formed from the pharmaceutically acceptable polymer.

In one aspect, the Compound (I) is in an amorphous form in the SDD. In one aspect, the Compound (I) is in free base form in the SDD.

In one aspect, in the spray-dried solid dispersions, the pharmaceutically acceptable polymer is hydroxypropyl methyl cellulose acetate succinate (HPMCAS).

In one aspect, in the spray-dried solid dispersions, the pharmaceutically acceptable polymer is hydroxypropyl methyl cellulose acetate succinate grade H (HPMCAS-H).

The spray-dried solid dispersion of the disclosure comprises at least about 1% by weight to at least about 25% by weight of 3,4-dimethyl-N-(2-phenyl-1H-pyrrolo[2,3-b]pyridin-5-yl)-1H-pyrazole-5-carboxamide (Compound I).

In some aspects, the spray-dried solid dispersion comprises at least about 1% by weight to at least about 5% by weight of Compound I.

In some aspects, the spray-dried solid dispersion comprises at least about 1% by weight to at least about 10% by weight of Compound I.

In some aspects, the spray-dried solid dispersion comprises at least about 1% by weight to at least about 15% by weight of Compound I.

In some aspects, the spray-dried solid dispersion comprises at least about 1% by weight to at least about 20% by weight of Compound I.

The spray-dried solid dispersion of the disclosure comprises at least about 75% by weight to at least about 99% by weight of the pharmaceutically acceptable polymer.

In some aspects, the spray-dried solid dispersion comprises at least about 80% by weight to at least about 99% by weight of the pharmaceutically acceptable polymer.

In some aspects, the spray-dried solid dispersion comprises at least about 85% by weight to at least about 99% by weight of the pharmaceutically acceptable polymer.

In some aspects, the spray-dried solid dispersion comprises at least about 90% by weight to at least about 99% by weight of the pharmaceutically acceptable polymer.

In some aspects, the spray-dried solid dispersion comprises at least about 95% by weight to at least about 99% by weight of the pharmaceutically acceptable polymer.

In the aspects of the disclosure, the spray-dried solid dispersion comprises hydroxypropyl methyl cellulose acetate succinate (HPMCAS) as the pharmaceutically acceptable polymer.

In the aspects of the disclosure, the spray-dried solid dispersion comprises hydroxypropyl methyl cellulose acetate succinate grade H (HPMCAS-H) as the pharmaceutically acceptable polymer.

In the spray-dried solid dispersion of the disclosure, the weight ratio of the 3,4-dimethyl-N-(2-phenyl-1H-pyrrolo[2,3-b]pyridin-5-yl)-1H-pyrazole-5-carboxamide (Compound I) to the pharmaceutically acceptable polymer is from about 1:3 to about 1:99.

In one aspect, the spray-dried solid dispersion comprises a solvent. The solvent is a combination of water and tetrahydrofuran (THF). The volume ratio of water to tetrahydrofuran is from about 1:2 to about 1:99. In one aspect, the solvent is THF.

### III. Pharmaceutical Formulations

In one aspect, provided herein is a pharmaceutical composition comprising Compound (I) and one or more pharmaceutically acceptable carriers, excipients or diluents. In one aspect, provided herein is a pharmaceutical composition comprising a spray-dried dispersion (SDD) comprising Compound (I) and a pharmaceutically acceptable polymer.

In some aspects, the Compound (I) in the pharmaceutical composition is in an amorphous and free base form.

In some aspects, the pharmaceutically acceptable polymer in the pharmaceutical composition is hydroxypropyl methyl cellulose acetate succinate grade (HPMCAS).

In some aspects, the pharmaceutically acceptable polymer in the pharmaceutical composition is hydroxypropyl methyl cellulose acetate succinate grade H (HPMCAS-H).

In one aspect, provided herein is a pharmaceutical composition comprising amorphous form of Compound (I), having not more than 5% (w/w) of any crystalline form, or no detectable amount of any crystalline form and one or more pharmaceutically acceptable carriers, excipients, or diluents.

Such pharmaceutical compositions, for example, can be in any oral dosage form such as, but not limited to a tablet, capsule, pill, powder, liquids, suspensions, emulsions, granules, sustained release formulations, solution, and suspension. In preferred embodiments, the pharmaceutical composition is an oral formulation, such a tablet suitable for single administration of precise dosages. Such dosage forms should allow the pharmaceutical composition to reach the target cells. Other factors are well known in the art and include considerations such as toxicity and dosage forms that retard the pharmaceutical composition from exerting its effects. Techniques and formulations generally may be found in Remington: The Science and Practice of Pharmacy, 21st edition, Lippincott, Williams and Wilkins, Philadelphia, Pa., 2005 (hereby incorporated by reference herein).

The methods and pharmaceutical compositions will typically be used in therapy for human subjects. However, they may also be used to treat similar or identical indications in other animal subjects.

The pharmaceutical compositions of the disclosure may be combined with one or more excipients. When a granulation processed is used, an excipient may be added prior to granulation (and thereby be intragranular) and/or may be added after granulation (and thereby be extragranular).

The excipients employed in the pharmaceutical compositions can impart good powder flow and compression characteristics to the material being compressed. It should be noted that excipients may serve multiple functions. Desirable characteristics of excipients can include high-compressibility's as to allow for strong tablets to be made at low compression forces; good powder flow properties that can improve the powder flow of other excipients in the composition; and cohesiveness, for example to prevent a tablet from crumbling during processing, shipping, and handling. Such properties are imparted to these excipients through pretreatment steps, such as, but not limited to dry granulation (e.g., by roller compaction, slugging), wet granulation, or spray drying spheronization (e.g., spray dried dispersion, solid nanodispersions). They may be classified according to the role that they play in the final tablet. Other excipients which give physical characteristics to a finished tablet are coloring and flavoring agents (e.g., in the case of chewable tablets). Examples of excipients are described, for example, in the Handbook of Pharmaceutical Excipients (5^{th} edition), edited by Raymond C. Rowe, Paul J. Sheskey, and Sian C. Owen; Publisher: Pharmaceutical Press.

In some embodiments, the pharmaceutical compositions will comprise pharmaceutically acceptable carriers or excipients, such as fillers, binders, disintegrants, glidants, lubricants, complexing agents, solubilizers, and surfactants, which may be chosen to facilitate administration of the compound by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-6-cyclodextrin), polysorbates (e.g. polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

The pharmaceutical composition provided herein can contain one or more fillers, which are added, for example, to increase the bulk weight of the blend resulting in a practical size for compression. Fillers that may be used include one or more of calcium salts such as calcium phosphate dibasic and sugars such as lactose, sucrose, dextrose, microcrystalline cellulose (MCC), mannitol, and maltodextrin. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. In some embodiments, the filler is microcrystalline cellulose, which can be manufactured by the controlled hydrolysis of alpha-cellulose. Suitable microcrystalline cellulose will have an average particle size of from about 20 nm to about 200 nm. Suitable microcrystalline cellulose can include Avicel® PH-101, Avicel® PH-102, Avicel® PH-103, Avicel® PH-105, Avicel® PH-113 and Avicel® PH-200, e.g., manufactured by FMC Corporation. In some embodiments, the one or more fillers include Avicel® PH-113 microcrystalline cellulose and Parteck® M 100 (Mannitol).

The pharmaceutical composition can also include one or more lubricants. The term “lubricant” as used herein is typically added to prevent the tableting materials from sticking to punches, minimize friction during tablet compression, and to allow for removal of the compressed tablet from the die. Examples of lubricants include, but are not limited to, colloidal silica, magnesium trisilicate, talc, magnesium carbonate, magnesium oxide, glycerylbehaptate, polyethylene glycol, ethylene oxide polymers (e.g., Carowax), sodium lauryl sulfate, magnesium stearate, aluminum stearate, calcium stearate, sodium stearyl fumarate, stearic acid, magnesium lauryl stearate, and mixtures of magnesium stearate with sodium lauryl sulfate. Exemplary lubricants include calcium stearate, magnesium stearate and sodium stearyl fumarate. In some embodiments, the one or more lubricants include sodium stearyl fumarate.

The pharmaceutical composition provided herein can also contain one or more glidants. The term “glidant” as used herein is a substance added to a powder that can improve its flowability, such as by reducing inter-particle friction. Exemplary glidants include but are not limited to colloidal silicas, colloidal silicon dioxide, fumed silica, CAB-O-SIL® M-5P, AEROSIL, talc, Syloid®, starch, and magnesium aluminum silicates. In some embodiments, the one or more glidants include colloidal silicon dioxide.

One or more disintegrants may be present in an amount necessary to expedite dissolution (e.g., increase the rate of tablet disintegration) in the pharmaceutical compositions provided herein. The term “disintegrant” as used herein refers to an excipient which can oppose the physical forces of particle bonding in a tablet or capsule when the oral formulation is placed in an aqueous environment. Disintegrants include starch derivatives and salts of carboxymethylcellulose. Examples of pharmaceutically acceptable disintegrants include, but are not limited to, starches, e.g., sodium starch glycolate, pregelatinized starch; clays; celluloses; alginates; gums; cross-linked polymers, e.g., cross-linked polyvinyl pyrrolidone (e.g., Polyplasdone™, polyvinyl polypyrrolidone, crospovidone), cross-linked calcium carboxymethylcellulose and cross-linked sodium carboxymethylcellulose (sodium croscarmellose); and soy polysaccharides. In some embodiments, the one or more disintegrants include Ac-Di-Sol® Croscarmellose Sodium that aids in the disintegration and drug dissolution of the tablet of the disclosure.

Also provided herein, is a pharmaceutical composition comprising a spray-dried solid dispersion comprising an amorphous form of Compound (I) and a pharmaceutically acceptable polymer, and wherein the 3,4-dimethyl-N-(2-phenyl-1H-pyrrolo[2,3-b]pyridin-5-yl)-1H-pyrazole-5-carboxamide (Compound I) is dispersed in a polymer matrix formed from the pharmaceutically acceptable polymer. In some embodiments, the pharmaceutically acceptable polymer is hydroxypropyl methyl cellulose acetate succinate grade H (HPMCAS-H).

### IV. Tablets

The pharmaceutical compositions may also be provided as tablets. Tablets may be uncoated, film, sugar coated, bisected, embossed, plain, layered, or sustained release. They can be made in a variety of sizes, shapes, and colors. Tablets may be swallowed, chewed, or dissolved in the buccal cavity or beneath the tongue.

In one embodiment, described herein is a tablet comprising the spray-dried solid dispersion comprising 3,4-dimethyl-N-(2-phenyl-1H-pyrrolo[2,3-b]pyridin-5-yl)-1H-pyrazole-5-carboxamide (Compound I), and a pharmaceutically acceptable polymer such as hydroxypropyl methyl cellulose acetate succinate grade H (HPMCAS-H), and one or more pharmaceutically acceptable ingredients selected from the group consisting of one or more binders, one or more buffering agents, one or more diluents, one or more disintegrants, one or more fillers, one or more film coatings, one or more lubricants, one or more glidants, and one or more surfactants.

In the tablet of the disclosure, the one or more pharmaceutically acceptable ingredients comprise colloidal silicon dioxide, croscarmellose sodium, sodium stearyl fumarate, mannitol, and microcrystalline cellulose.

The tablet of the disclosure comprises at least about 1% by weight to at least about 20% by weight of 3,4-dimethyl-N-(2-phenyl-1H-pyrrolo[2,3-b]pyridin-5-yl)-1H-pyrazole-5-carboxamide (Compound I).

In some aspects, the tablet comprises at least about 5% by weight to at least about 20% by weight of Compound I.

In some aspects, the tablet comprises at least about 15% by weight to at least about 20% by weight of Compound I.

The tablet of the disclosure comprises at least about 10% by weight to at least about 90% by weight of the pharmaceutically acceptable polymer.

The tablet of the disclosure comprises at least about 20% by weight to at least about 90% by weight of the pharmaceutically acceptable polymer.

The tablet of the disclosure comprises at least about 30% by weight to at least about 90% by weight of the pharmaceutically acceptable polymer.

In some aspects, the tablet comprises at least about 40% by weight to at least about 90% by weight of the pharmaceutically acceptable polymer.

In some aspects, the tablet comprises at least about 50% by weight to at least about 90% by weight of the pharmaceutically acceptable polymer.

In some aspects, the tablet comprises at least about 60% by weight to at least about 90% by weight of the pharmaceutically acceptable polymer.

In some aspects, the tablet comprises at least about 70% by weight to at least about 90% by weight of the pharmaceutically acceptable polymer.

In some aspects, the tablet comprises at least about 80% by weight to at least about 90% by weight of the pharmaceutically acceptable polymer.

In the tablet of the disclosure, the pharmaceutically acceptable polymer is hydroxypropyl methyl cellulose acetate succinate grade (HPMCAS).

In the tablet of the disclosure, the pharmaceutically acceptable polymer is hydroxypropyl methyl cellulose acetate succinate grade H (HPMCAS-H).

The tablet of the disclosure comprises at least about 3% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

The tablet of the disclosure comprises at least about 5% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 10% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 15% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 20% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 25% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 30% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 35% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 40% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 45% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 50% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 55% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

In some aspects, the tablet comprises at least about 60% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

The disclosure also relates to a tablet comprising at least about 1% by weight to at least about 20% by weight of 3,4-dimethyl-N-(2-phenyl-1H-pyrrolo[2,3-b]pyridin-5-yl)-1H-pyrazole-5-carboxamide (Compound I), at least about 10% by weight to at least about 90% by weight of hydroxypropyl methyl cellulose acetate succinate grade H (HPMCAS-H), and at least about 3% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

Tablets may be presented in unit dose forms containing a predetermined amount of active ingredient per unit dose. Such a unit may contain, for example, 0.5 mg to 1 g, preferably 1 mg to 700 mg, more preferably 5 mg to 100 mg of Compound (I), depending on the condition being treated, the route of administration, and the age, weight and condition of the patient. Preferred unit dosage formulations are those containing a daily dose, weekly dose, monthly dose, a sub-dose or an appropriate fraction thereof, of an active ingredient. Furthermore, such pharmaceutical formulations may be prepared by any of the methods well known in the pharmacy art.

In some aspects, the tablet of the disclosure is taken once daily.

In other aspects, the tablet of the disclosure is taken twice daily.

In yet another aspect, the tablet of the disclosure is taken continuously in 28-day cycles.

The unit dose to be administered can be determined by standard procedures taking into account factors such as the activity of API (in vitro, e.g. the compound IC₅₀ vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Generally, a dose will be in the range of about 0.01 to 50 mg/kg, also about 0.1 to 20 mg/kg of the subject being treated. Multiple doses may be used.

The pharmaceutical compositions described herein may also be used in combination with other therapies for treating the same disease. Such combination use includes administration of the compounds and one or more other therapeutics at different times, or co-administration of the compound and one or more other therapies. In some embodiments, dosage may be modified for pharmaceutical compositions of the disclosure or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than the pharmaceutical compositions described herein, or at the same time as the pharmaceutical compositions described herein. Use in combination also includes use with a therapy or medical procedure that is administered once or infrequently, such as surgery, along with the pharmaceutical compositions described herein administered within a short time or longer time before or after the other therapy or procedure. In some embodiments, the present disclosure provides for delivery of the pharmaceutical compositions described herein and one or more other drug therapeutics delivered by a different route of administration or by the same route of administration. The use in combination for any route of administration includes delivery of the pharmaceutical compositions described herein and one or more other drug therapeutics delivered by the same route of administration together in any formulation, including formulations where the two compounds are chemically linked in such a way that they maintain their therapeutic activity when administered. In one aspect, the other drug therapy may be co-administered with the pharmaceutical compositions described herein. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of a compound described herein and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

Provided herein, is an embodiment for a method of treating a subject suffering from disease or condition selected from Acute Myeloid Leukemia (AML), Gastrointestinal Stromal Tumors (GIST), Mast Cell Leukemia (MCL) and mastocytosis comprising administering orally to the subject the tablet of the disclosure.

Also provided herein, in another embodiment, is a use of the pharmaceutical composition or tablets described herein for the preparation of medicament for the treatment of disease or condition selected from Acute Myeloid Leukemia (AML), Gastrointestinal Stromal Tumors (GIST), Mast Cell Leukemia (MCL) and mastocytosis.

### V. Disease Indications and Modulations of c-Kit Kinase

A. Exemplary Diseases Associated with c-Kit or Mutant Form of c-Kit

The formulations described herein are useful for treating disorders related to c-Kit e.g., diseases related to unregulated kinase signal transduction, including cell proliferative disorders, fibrotic disorders and metabolic disorders, among others. As described in more detail below and in Lipson et al., U.S. 20040002534 (U.S. application Ser. No. 10/600,868, filed Jun. 23, 2003) which is incorporated herein by reference in its entirety, cell proliferative disorders which can be treated by the present disclosure include cancers, and mast cell proliferative disorders.

The presence of c-Kit or mutant c-Kit has also been associated with a number of different types of cancers, diseases and conditions, as described below. In addition, the association between abnormalities in c-Kit and disease are not restricted to cancer. As such, c-Kit has been associated with malignancies, including mast cell tumors, small cell lung cancer, testicular cancer, gastrointestinal stromal tumors (GISTs), metastatic GISTs, glioblastoma, astrocytoma, neuroblastoma, carcinomas of the female genital tract, sarcomas of neuroectodermal origin, colorectal carcinoma, carcinoma in situ, Schwann cell neoplasia associated with neurofibromatosis, acute myelocytic leukemia (AML), acute lymphocytic leukemia, chronic myelogenous leukemia, mastocytosis, melanoma (mucosal and cutaneous), thyroid carcinoma, breast cancer, germ cell tumors, including mixed germ cell tumors, ovarian germ cell tumors, dysgerminomas, seminomas, large cell neuroendocrine carcinoma, prostate cancer, and canine mast cell tumors, and inflammatory diseases, including asthma, rheumatoid arthritis, allergic rhinitis, multiple sclerosis, inflammatory bowel syndrome, transplant rejection, hypereosinophilia, urticaria pigmentosa (UP), telangiectasia macularis eruptiva perstans (TMEP), systemic mastocytosis, advanced systemic mastocytosis (AdvSM), systemic mastocytosis with associated hematological neoplasm (SM-AHN), nonadvanced systemic mastocytosis (NonAdvSM), indolent systemic (ISM), cutaneous mastocytosis (CM), smoldering systemic mastocytosis (SSM), aggressive systemic, mast cell leukemia, mast cell sarcoma, mast cell activation syndrome (MCAS), maculopapular cutaneous mastocytosis/urticaria pigmentosa, chronic myeloid leukemia (CML), myelodysplastic syndrome (MDS), myelofibrosis, and sinonasal lymphomas. The presence of mutant forms of c-Kit has been associated with diseases or conditions, for example, gastrointestinal stromal tumors (GISTs), mast cell leukemia, germ-cell tumor, t-cell lymphoma, mastocytosis, acute lymphocytic leukemia and seminama.

B. Exemplary Malignant Diseases Associated with c-Kit

Aberrant expression and/or activation of c-Kit and/or mutant form of c-Kit has been implicated in a variety of cancers (Roskoski, 2005, Biochemical and biophysical Research Comm. 338: 1307-1315). Evidence for a contribution of c-Kit to neoplastic pathology includes its association with leukemias and mast cell tumors, small cell lung cancer, testicular cancer, and some cancers of the gastrointestinal tract and central nervous system. In addition, c-Kit has been implicated in playing a role in carcinogenesis of the female genital tract (Inoue, et al., 1994, Cancer Res. 54(11):3049-3053), sarcomas of neuroectodermal origin (Ricotti, et al., 1998, Blood 91:2397-2405), and Schwann cell neoplasia associated with neurofibromatosis (Ryan, et al., 1994, J. Neuro. Res. 37:415-432). It was found that mast cells are involved in modifying the tumor microenvironment and enhancing tumor growth (Yang et al., 2003, J Clin Invest. 112:1851-1861; Viskochil, 2003, J Clin Invest. 112:1791-1793). Thus, c-Kit is a useful target in treating neurofibromatosis as well as malignant tumors.

Small cell lung carcinoma: c-Kit kinase receptor has been found to be aberrantly expressed in many cases of small cell lung carcinoma (SCLC) cells (Hibi, et al., 1991, Oncogene 6:2291-2296). Thus, as an example, inhibition of c-Kit kinase can be beneficial in treatment of SCLC, e.g., to improve the long term survival of patients with SCLC.

Leukemias: SCF binding to the c-Kit protects hematopoietic stem and progenitor cells from apoptosis (Lee, et al., 1997, J. Immunol. 159:3211-3219), thereby contributing to colony formation and hematopoiesis. Expression of c-Kit is frequently observed in acute myelocytic leukemia (AML), and in some cases of acute lymphocytic leukemia (ALL) (for reviews, see Sperling, et al., 1997, Haemat 82:617-621; Escribano, et al., 1998, Leuk. Lymph. 30:459-466). Although c-Kit is expressed in the majority of AML cells, its expression does not appear to be prognostic of disease progression (Sperling, et al, 1997, Haemat 82:617-621). However, SCF protected AML cells from apoptosis induced by chemotherapeutic agents (Hassan, et al., 1996, Acta. Hem. 95:257-262). Inhibition of c-Kit by the present disclosure will enhance the efficacy of these agents and can induce apoptosis of AML cells.

The clonal growth of cells from patients with myelodysplastic syndrome (Sawada, et al., 1996, Blood 88:319-327) or chronic myelogenous leukemia (CML) (Sawai, et al., 1996, Exp. Hem. 2:116-122) was found to be significantly enhanced by SCF in combination with other cytokines. CML is characterized by expansion of Philadelphia chromosome positive cells of the marrow (Verfaillie, et al., Leuk. 1998, 12:136-138), which appears to primarily result from inhibition of apoptotic death (Jones, Curr. Opin. Onc. 1997, 9:3-7). The product of the Philadelphia chromosome, p210^{BCR-ABL}, has been reported to mediate inhibition of apoptosis (Bedi, et al., Blood 1995, 86:1148-1158). Since p210BCR-ABL and c-Kit both inhibit apoptosis and p62dok has been suggested as a substrate (Carpino, et al., Cell 1997, 88:197-204), clonal expansion mediated by these kinases may occur through a common signaling pathway. However, c-Kit has also been reported to interact directly with p210^{BCR-ABL}(Hallek, et al., Brit. J Haem. 1996, 94:5-16), which suggests that c-Kit has a more causative role in CML pathology. Therefore, inhibition of c-Kit will be useful in the treatment of the above disorders.

Gastrointestinal cancers: Normal colorectal mucosa does not express c-Kit (Bellone, et al., 1997, J. Cell Physiol. 172:1-11). However, c-Kit is frequently expressed in colorectal carcinoma (Bellone, et al., 1997, J. Cell Physiol. 172: 1-11), and autocrine loops of SCF and c-Kit have been observed in several colon carcinoma cell lines (Toyota, et al., 1993, Turn Biol 14:295-302; Lahm, et al., 1995, Cell Growth & Differ 6:1111-1118; Bellone, et al., 1997, J. Cell Physiol. 172:1-11). Furthermore, disruption of the autocrine loop by the use of neutralizing antibodies (Lahm, et al., 1995, Cell Growth & Differ. 6:1111-1118) and down regulation of c-Kit and/or SCF significantly inhibits cell proliferation (Lahm, et al., 1995, Cell Growth & Differ 6:1111-1118; Bellone, et al., 1997, J. Cell Physiol. 172:1-11).

SCF/c-Kit autocrine loops have been observed in gastric carcinoma cell lines (Turner, et al., 1992, Blood 80:374-381; Hassan, et al., 1998, Digest. Dis. Science 43:8-14), and constitutive c-Kit activation also appears to be important for gastrointestinal stromal tumors (GISTs). GISTs are the most common mesenchymal tumor of the digestive system. More than 90% of GISTs express c-Kit, which is consistent with the putative origin of these tumor cells from interstitial cells of Cajal (ICCs) (Hirota, et al., 1998, Science 279:577-580). ICCs are thought to regulate contraction of the gastrointestinal tract, and patients lacking c-Kit in their ICCs exhibited a myopathic form of chronic idiopathic intestinal pseudo-obstruction (Isozaki, et al., 1997, Amer. J. of Gast. 9 332-334). The c-Kit expressed in GISTs from several different patients was observed to have mutations in the intracellular juxtamembrane domain leading to constitutive activation of c-Kit (Hirota, et al., 1998, Science 279:577-580). Hence, inhibition of c-Kit kinase will be an efficacious means for the treatment of these cancers.

Overexpression or constitutive activation of Kit mutations have been implicated and associated in gastrointestinal stromal tumors (GISTs) and most GISTs contain oncogenic KIT receptor or PDGFRA receptor tyrosine kinase mutations (Miettinen, et al., 2006, Arch Pathol Lab Med, 130: 14661478; Fletcher, et al., 2007, Current Opinion in Genetics & Development, 17:3-7; and Frost, et al. 2002, Molecular Cancer Therapeutics, 1:1115-1124). Frost, et al, 2002 has shown that D816V KIT mutation is resistant to imatinib, such that additional types of c-Kit inhibitors are useful. Many GISTs have activating mutations in the KIT justamembrane regions (Lux, et al., 2000, American Journal Pathology, 156:795). Constitutive activation of the Kit receptor tyrosine kinase is a central pathogenic event in most GISTs and generally results from oncogenic point mutations (Heinrich, et al. 2002, Human Pathology, 33:484-495). Inhibition of wild-type KIT and/or certain mutant KIT isoforms with a small molecule tyrosine kinase inhibitor has become standard of care for treating patient with metastatic GISTs (Schittenhelm, et al. 2006, Cancer Res., 66: 473-481). Therefore, inhibition of c-Kit kinase and/or mutant c-Kit kinase will be an efficacious means for the treatment of GISTs.

Testicular cancers: Male germ cell tumors have been histologically categorized into seminomas, which retain germ cell characteristics, and nonseminomas which can display characteristics of embryonal differentiation. Both seminomas and nonseminomas are thought to initiate from a preinvasive stage designated carcinoma in situ (CIS) (Murty, et al., 1998, Sem. Oncol. 25:133-144). Both c-Kit and SCF have been reported to be essential for normal gonadal development during embryogenesis (Loveland, et al., 1997, J. Endocrinol 153:337-344). Loss of either the receptor or the ligand resulted in animals devoid of germ cells. In postnatal testes, c-Kit has been found to be expressed in Leydig cells and spermatogonia, while SCF was expressed in Sertoli cells (Loveland, et al., 1997, J. Endocrinol 153:337-344). Testicular tumors develop from Leydig cells with high frequency in transgenic mice expressing human papilloma virus 16 (HPV16) E6 and E7 oncogenes (Kondoh, et al., 1991, J. Virol. 65:3335-3339; Kondoh, et al., 1994, J. Urol. 152:2151-2154). These tumors express both c-Kit and SCF, and an autocrine loop may contribute to the tumorigenesis (Kondoh, et al., 1995, Oncogene 10:341-347) associated with cellular loss of functional p53 and the retinoblastoma gene product by association with E6 and E7 (Dyson, et al., 1989, Science 243:934-937; Werness, et al., 1990, Science 248:76-79; Scheffner, et al., 1990, Cell 63:1129-1136). Defective signaling mutants of SCF (Kondoh, et al., 1995, Oncogene 10:341-347) or c-kit (Li, et al., 1996, Canc. Res. 56:4343-4346) inhibited formation of testicular tumors in mice expressing HPV16 E6 and E7. The c-kit kinase activation is pivotal to tumorigenesis in these animals and thus modulation of the c-kit kinase pathway by the present disclosure will prevent or treat such disorders.

Expression of c-kit in germ cell tumors shows that the receptor is expressed by the majority of carcinomas in situ and seminomas, but c-kit is expressed in only a minority of nonseminomas (Strohmeyer, et al., 1991, Canc. Res. 51:1811-1816; Rajpert-de Meyts, et al., 1994, Int. J. Androl. 17:85-92; Izquierdo, et al., 1995, J. Pathol. 177:253-258; Strohmeyer, et al., 1995, J. Urol. 153:511-515; Bokenmeyer, et al., 1996, J. Cancer Res. Clin. Oncol. 122:301-306; Sandlow, et al., 1996, J. Androl. 17:403-408). Therefore, inhibition of c-kit kinase provides a means for treating these disorders.

CNS cancers: SCF and c-kit are expressed throughout the CNS of developing rodents, and the pattern of expression indicates a role in growth, migration and differentiation of neuroectodermal cells. Expression of both receptor and ligand have also been reported in the adult brain (Hamel, et al., 1997, J. Neuro-Onc. 35:327-333). Expression of c-kit has also been observed in normal human brain tissue (Tada, et al. 1994, J. Neuro 80:1063-1073). Glioblastoma and astrocytoma, which define the majority of intracranial tumors, arise from neoplastic transformation of astrocytes (Levin, et al., 1997, Principles & Practice of Oncology: 2022-2082). Expression of c-kit has been observed in glioblastoma cell lines and tissues (Berdel, et al., 1992, Canc. Res. 52:3498-3502; Tada, et al. 1994, J. Neuro 80:1063-1073; Stanulla, et al., 1995, Act Neuropath 89:158-165).

Cohen, et al., 1994, Blood 84:3465-3472 reported that all 14 neuroblastoma cell lines examined contained c-kit/SCF autocrine loops, and expression of both the receptor and ligand were observed in 45% of tumor samples examined. In two cell lines, anti-c-kit antibodies inhibited cell proliferation, suggesting that the SCF/c-kit autocrine loop contributed to growth (will Cohen, et al., 1994, Blood 84:3465-3472). Hence, c-kit kinase inhibitors can be used to treat these cancers.

C. Exemplary Mast Cell Diseases Involving c-Kit

Excessive activation of c-kit is also associated with diseases resulting from an overabundance of mast cells. Mastocytosis is the term used to describe a heterogeneous group of disorders characterized by excessive mast cell proliferation (Metcalfe, 1991, J. Invest. Derm 93:2S-4S; Golkar, et al., 1997, Lancet 349:1379-1385). Elevated c-kit expression was reported on mast cells from patients with aggressive mastocytosis (Nagata, et al., 1998, Leukemia 12:175-181).

Additionally, mast cells and eosinophils represent key cells involved in allergy, inflammation and asthma (Thomas, et al., 1996, Gen. Pharmacol 27:593-597; Metcalfe, et al., 1997, Physiol Rev 77:1033-1079; Naclerio, et al., 1997, JAMA 278:1842-1848; Costa, et al., 1997, JAMA 278:1815-1822). SCF, and hence c-kit, directly and indirectly regulates activation of both mast cells and eosinophils, thereby influencing the primary cells involved in allergy and asthma through multiple mechanisms. Because of this mutual regulation of mast cell and eosinophil function, and the role that SCF can play in this regulation, inhibition of c-kit can be used to treat allergy-associated chronic rhinitis, inflammation and asthma.

Mastocytosis: SCF (also known as mast cell growth factor) stimulation of c-kit has been reported to be essential for the growth and development of mast cells (Hamel, et al., 1997, J. Neuro-Onc. 35:327-333; Kitamura, et al., 1995, Int. Arch. Aller. Immunol. 107:54-56). Mice with mutations of c-kit that attenuate its signaling activity have exhibited significantly fewer mast cells in their skin (Tsujimura, 1996, Pathol Int 46:933-938). Excessive activation of c-kit can be associated with diseases resulting from an overabundance of mast cells.

Mastocytosis is limited to the skin in the majority of patients, but can involve other organs in 15-20% of patients (Valent, 1996, Wein/Klin Wochenschr 108:385-397; Golkar, et al., 1997, Lancet 349:1379-1385). Even among patients with systemic mastocytosis, the disease can range from having a relatively benign prognosis to aggressive mastocytosis and mast cell leukemia. (Valent, 1996, Wein/Klin Wochenschr 108:385-397; Golkar, et al., 1997, Lancet 349:1379-1385). c-kit has been observed on malignant mast cells from canine mast cell tumors (London, et al., 1996, J. Compar. Pathol. 115:399-414), as well as on mast cells from patients with aggressive systemic mastocytosis (Baghestanian, et al., 1996, Leuk.:116-122; Castells, et al., 1996, J. Aller. Clin. Immunol. 98:831-840).

SCF has been shown to be expressed on stromal cells as a membrane-bound protein, and its expression can be induced by fibrogenic growth factors such as PDGF. It has also been shown to be expressed on keratinocytes as a membrane-bound protein in normal skin. However, in the skin of patients with mastocytosis, an increased amount of soluble SCF has been observed (Longley, et al., 1993, New Engl. J. Med. 328:1302-1307).

Mast cell chymase has been reported to cleave membrane-associated SCF to a soluble and biologically active form. This mast cell-mediated process can generate a feedback loop to enhance mast cell proliferation and function (Longley, et al., 1997, Proc. Natl. Acad. Sci. 94:9017-9021), and may be important for the etiology of mastocytosis. Transgenic mice overexpressing a form of SCF that could not be proteolytically released from keratinocytes did not develop mastocytosis, while similar animals expressing normal SCF in keratinocytes exhibited a phenotype resembling human cutaneous mastocytosis (Kunisada, et al., 1998, J. Exp. Med. 187:1565-1573). Formation of large amounts of soluble SCF can contribute to the pathology associated with mastocytosis in some patients and the present disclosure can treat or prevent such disorders by modulating the interaction between SCF and c-kit kinase. Several different mutations of c-kit that resulted in constitutive kinase activity have been found in human and rodent mast cell tumor cell lines (Furitsu, et al., 1993, J. Clin. Invest. 92:1736-1744; Tsujimura, et al., 1994, Blood 9:2619-2626; Tsujimura, et al., 1995, Int. Arch. Aller. Immunol 106:377-385; Tsujimura, 1996, Pathol Int 46:933-938). In addition, activating mutations of the c-kit gene have been observed in peripheral mononuclear cells isolated from patients with mastocytosis and associated hematologic disorders (Nagata, et al., 1998, Mastocytosis Leuk 12:175-181), and in mast cells from a patient with urticaria pigmentosa and aggressive mastocytosis (Longley, et al., 1996, Nat. Gen. 12:312-314). Inhibition of c-kit kinase will therefore prove to have an excellent therapeutic role in the treatment of these disorders.

In some patients, activating mutations of c-kit may be responsible for the pathogenesis of the disease and these patients can be treated, or their diseases prevented, by modulation of the SCF interaction with c-kit kinase. SCF activation of c-kit has been shown to prevent mast cell apoptosis which may be critical for maintaining cutaneous mast cell homeostasis (Iemura, et al., 1994, Amer. J. Pathol 144:321-328; Yee, et al., 1994, J. Exp. Med. 179:1777-1787; Mekori, et al., 1994, J. Immunol 153:2194-2203; Mekori, et al., 1995, Int. Arch. Allergy Immunol. 107:137-138). Inhibition of mast cell apoptosis can lead to the mast cell accumulation associated with mastocytosis. Thus, observation of c-kit activation resulting from overexpression of the receptor, excessive formation of soluble SCF, or mutations of the c-kit gene that constitutively activate its kinase, provides a rationale that inhibition of the kinase activity of c-kit will decrease the number of mast cells and provide benefit for patients with mastocytosis.

For cells with activating c-kit mutations, it was found that inhibitors of c-kit inhibit or even kill the cells (Ma et al., 2000, J Invest Dermatol. 114:392-394), particularly for mutations in the regulatory region (Ma et al., 2002, Blood 99:1741-1744). Ma et al., 2002, also showed that for mutations in the catalytic region, inhibitors STI571 (Gleevec) and SU9529 did not inhibit the cells, such that additional types of c-kit inhibitors are useful. Thus, c-kit inhibitors can be used against both wild-type c-kit as well as c-kit having mutations, e.g., activating mutations in the regulatory region and/or catalytic region.

It has been shown that mastocytosis is characterized by a pathologic increase of mast cells in tissues associated with mutations in KIT (Metcalfe, 2008, Blood, 112:946-956; and Ma, et al., 2002). D816 mutation of c-kit has been detected in patients with mastocytosis (Taylor, et al., 2001, Blood, 98:1195-1199; and Longley, et al. 1999, Proc. Natl. Acad. Sci. 96:1609-14). Inhibition of KIT oncogenic protein KITD816V with small molecule tyrosine kinase inhibitor is capable of treating patients with systemic mastocytosis (Shah, et al., 2006, Blood, 108:286-291). Thus, c-kit inhibitors can be used in treating patients with mastocytosis.

Asthma & Allergy: Mast cells and eosinophils represent key cells in parasitic infection, allergy, inflammation, and asthma (Thomas, et al., 1996, Gen. Pharmacol 27:593-597; Metcalfe, et al., 1997, Physiol Rev 77:1033-1079; Holgate, 1997, CIBA Found. Symp.; Naclerio, et al, 1997, JAMA 278:1842-1848; Costa, et al., 1997, JAMA 778:1815-1822). SCF has been shown to be essential for mast cell development, survival and growth (Kitamura, et al., 1995, Int. Arch. Aller. Immunol. 107:54-56; Metcalfe, et al., 1997, Physiol Rev 77:1033-1079). In addition, SCF cooperates with the eosinophil-specific regulator, IL-5, to increase the development of eosinophil progenitors (Metcalf, et al., 1998, Proc. Natl. Acad. Sci., USA 95:6408-6412). SCF has also been reported to induce mast cells to secrete factors (Okayama, et al., 1997, Int. Arch. Aller. Immunol. 114:75-77; Okayama, et al., 1998, Eur. J. Immunol. 28:708-715) that promote the survival of eosinophils (Kay, et al., 1997, Int. Arch. Aller. Immunol. 113:196-199), which may contribute to chronic, eosinophil-mediated inflammation (Okayama, et al., 1997, Int. Arch. Aller. Immunol. 114:75-77; Okayama, et al., 1998, Eur. J. Immunol. 28:708-715). In this regard, SCF directly and indirectly regulates activation of both mast cells and eosinophils.

SCF induces mediator release from mast cells, as well as priming these cells for IgE-induced degranulation (Columbo, et al., 1992, J. Immunol 149:599-602) and sensitizing their responsiveness to eosinophil-derived granule major basic protein (Furuta, et al., 1998, Blood 92:1055-1061). Among the factors released by activated mast cells are IL-5, GM-CSF and TNF-□, which influence eosinophil protein secretion (Okayama, et al., 1997, Int. Arch. Aller. Immunol. 114:75-77; Okayama, et al., 1998, Eur. J. Immunol. 28:708-715). In addition to inducing histamine release from mast cells (Luckacs, et al., 1996, J. Immunol. 156:3945-3951; Hogaboam, et al., 1998, J. Immunol. 160:6166-6171), SCF promotes the mast cell production of the eosinophil chemotactic factor, eotaxin (Hogaboam, et al., 1998, J. Immunol. 160:6166-6171), and eosinophil infiltration (Luckacs, et al., 1996, J. Immunol. 156:3945-3951).

SCF also directly influences the adhesion of both mast cells (Dastych, et al., 1994, J. Immunol. 152:213-219; Kinashi, et al., 1994, Blood 83:1033-1038) and eosinophils (Yuan, et al., 1997, J. Exp. Med. 186:313-323), which in turn, regulates tissue infiltration. Thus, SCF can influence the primary cells involved in allergy and asthma through multiple mechanisms. Currently, corticosteroids are the most effective treatment for chronic rhinitis and inflammation associated with allergy (Naclerio, et al., 1997, JAMA 278:1842-1848; Meltzer, 1997, Aller. 52:33-40). These agents work through multiple mechanisms including reduction of circulating and infiltrating mast cells and eosinophils, and diminished survival of eosinophils associated with inhibition of cytokine production (Meltzer, 1997, Aller. 52:33-40). Steroids have also been reported to inhibit the expression of SCF by fibroblasts and resident connective tissue cells, which leads to diminished mast cell survival (Finotto, et al., 1997, J. Clin. Invest. 99 1721-1728). Because of the mutual regulation of mast cell and eosinophil function, and the role that SCF can play in this regulation, inhibition of c-kit kinase will provide a means to treat allergy-associated chronic rhinitis, inflammation and asthma.

Inflammatory arthritis (e.g. rheumatoid arthritis): Due to the association of mast cells with the arthritic process (Lee et al., 2002, Science 297:1689-1692), c-kit provides a useful target for prevention, delay, and/or treatment of inflammatory arthritis, such as rheumatoid arthritis.

Multiple sclerosis: Mast cells have been shown to play an extensive role in autoimmune diseases, as demonstrated in the mouse model of multiple sclerosis (MS), experimental allergic encephalomyelitis (EAE). Mast cells were indicated to be required for full manifestation of the disease. Secor et al., 2000, J Exp Med 191:813-821. Thus, c-kit also provides a useful target for the prevention, delay, and/or treatment of multiple sclerosis.

### VI. Methods of Treating Conditions Mediated by c-Kit Kinase

In another aspect, the present disclosure provides a method for treating a subject suffering from or at risk of a c-kit and or a mutant c-kit protein kinase mediated diseases or conditions. The method includes orally administering to the subject an effective amount of a tablet disclosed herein.

In some embodiments, the mutant c-kit kinase has a mutation selected from D816F, D816H, D816N, D816Y, D816V, K642E, Y823D, Del 550-558, Del 557-561, N822K, V654A, N822H, Del 550-558+V654A, Del 557-561+V654A, Ins503AY, V560G, 558NP, Del 557-558, Del W559-560, F522C, Del 579, R634W, K642E, T801I, C809G, D820Y, N822K, N822H, Y823D, Y823C or T670I or combinations thereof. In one embodiment, the mutant c-kit has an activating D816 mutation. In one embodiment, the mutant c-kit has an activating D816V mutation. In another embodiment, the mutant c-kit has a V560G mutation. In yet another embodiment, the mutant c-kit has an activating D816V and V560G mutations. In certain embodiments, the method involves administering to the subject an effective amount of a tablet as described herein in combination with one or more other therapies for the disease or condition.

In some embodiments, the disclosure provides a method of suppressing undesired proliferation of tumor cells expressing a D816 (such as D816F, D816H, D816N, D816Y or D816V) and/or V560G mutant c-kit protein kinase. The method includes contacting tumor cells expressing D816 (such as D816F, D816H, D816N, D816Y or D816V) and/or V560G mutant c-kit protein kinase with an effective amount of a tablet as described herein. In some instances, the tumor cells expressing D816V and/or V560G mutant c-kit kinase.

In certain embodiments, the disclosure provides a method of treating a c-kit protein kinase D816 (such as D816F, D816H, D816N, D816Y or D816V) and/or V560G mutation-positive patient. The method includes administering to the patient in need thereof an effective amount of a tablet as described herein. In some embodiments, the patient is D816V mutation-positive. In other embodiments, the patient is V560G mutation-positive. In some embodiments, the patient is D816V and V560G mutation-positive. In certain instances, the patient is suffering from gastrointestinal stromal tumors (GISTs) and/or mastocytosis.

In some embodiments, the diseases or conditions treatable with the compounds of the present disclosure include, but are not limited to, multi-infarct dementia, head injury, spinal cord injury, Alzheimer's disease (AD), Parkinson's disease, seizures and epilepsy; neoplastic diseases including, but not limited to, melanoma, glioma, glioblastoma multiforme, pilocytic astrocytoma, sarcoma, carcinoma (e.g. gastrointestinal, liver, biliary tract, bile duct (cholangiocarcinoma), colorectal, lung, gallbladder, breast, pancreatic, thyroid, renal, ovarian, adrenocortical, prostate), lymphoma (e.g. histiocytic lymphoma) neurofibromatosis, gastrointestinal stromal tumors, acute myeloid leukemia, myelodysplastic syndrome, leukemia, tumor angiogenesis, neuroendocrine tumors such as medullary thyroid cancer, carcinoid, small cell lung cancer, Kaposi's sarcoma, and pheochromocytoma; pain of neuropathic or inflammatory origin, including, but not limited to, acute pain, chronic pain, cancer-related pain, and migraine; cardiovascular diseases including, but not limited to, heart failure, ischemic stroke, cardiac hypertrophy, thrombosis (e.g. thrombotic microangiopathy syndromes), atherosclerosis, and reperfusion injury; inflammation and/or proliferation including, but not limited to, psoriasis, eczema, arthritis and autoimmune diseases and conditions, osteoarthritis, endometriosis, scarring, vascular restenosis, fibrotic disorders, rheumatoid arthritis, inflammatory bowel disease (IBD); immunodeficiency diseases, including, but not limited to, organ transplant rejection, graft versus host disease, and Kaposi's sarcoma associated with HIV; renal, cystic, or prostatic diseases, including, but not limited to, diabetic nephropathy, polycystic kidney disease, nephrosclerosis, glomerulonephritis, prostate hyperplasia, polycystic liver disease, tuberous sclerosis, Von Hippel Lindau disease, medullary cystic kidney disease, nephronophthisis, and cystic fibrosis; metabolic disorders, including, but not limited to, obesity; infection, including, but not limited to Helicobacter pylori, Hepatitis and Influenza viruses, fever, HIV, and sepsis; pulmonary diseases including, but not limited to, chronic obstructive pulmonary disease (COPD) and acute respiratory distress syndrome (ARDS); genetic developmental diseases, including, but not limited to, Noonan's syndrome, Costello syndrome, (faciocutaneoskeletal syndrome), LEOPARD syndrome, cardio-faciocutaneous syndrome (CFC), and neural crest syndrome abnormalities causing cardiovascular, skeletal, intestinal, skin, hair and endocrine diseases; and diseases associated with muscle regeneration or degeneration, including, but not limited to, sarcopenia, muscular dystrophies (including, but not limited to, Duchenne, Becker, Emery-Dreifuss, Limb-Girdle, Facioscapulohumeral, Myotonic, Oculopharyngeal, Distal and Congenital Muscular Dystrophies), motor neuron diseases (including, but not limited to, amyotrophic lateral sclerosis, infantile progressive spinal muscular atrophy, intermediate spinal muscular atrophy, juvenile spinal muscular atrophy, spinal bulbar muscular atrophy, and adult spinal muscular atrophy), inflammatory myopathies (including, but not limited to, dermatomyositis, polymyositis, and inclusion body myositis), diseases of the neuromuscular junction (including, but not limited to, myasthenia gravis, Lambert-Eaton syndrome, and congenital myasthenic syndrome), myopathies due to endocrine abnormalities (including, but not limited to, hyperthyroid myopathy and hypothyroid myopathy) diseases of peripheral nerve (including, but not limited to, Charcot-Marie-Tooth disease, Dejerine-Sottas disease, and Friedreich's ataxia), other myopathies (including, but not limited to, myotonia congenita, paramyotonia congenita, central core disease, nemaline myopathy, myotubular myopathy, and periodic paralysis), and metabolic diseases of muscle (including, but not limited to, phosphorylase deficiency, acid maltase deficiency, phosphofructokinase deficiency, debrancher enzyme deficiency, mitochondrial myopathy, carnitine deficiency, carnitine palmatyl transferase deficiency, phosphoglycerate kinase deficiency, phosphoglycerate mutase deficiency, lactate dehydrogenase deficiency, and myoadenylate deaminase deficiency). In one embodiment, the disease or condition is selected from the group consisting of melanoma, glioma, glioblastoma multiforme, pilocytic astrocytoma, sarcoma, liver cancer, biliary tract cancer, cholangiocarcinoma, colorectal cancer, lung cancer, gallbladder cancer, breast cancer, pancreatic cancer, thyroid cancer, renal cancer, ovarian cancer, adrenocortical cancer, prostate cancer, histiocytic lymphoma, neurofibromatosis, gastrointestinal stromal tumors, acute myeloid leukemia, myelodysplastic syndrome, leukemia, tumor angiogenesis, medullary thyroid cancer, carcinoid, small cell lung cancer, Kaposi's sarcoma, pheochromocytoma, acute pain, chronic pain, and polycystic kidney disease. In a preferred embodiment, the disease or condition is selected from the group consisting of melanoma, glioma, glioblastoma multiforme, pilocytic astrocytoma, colorectal cancer, thyroid cancer, lung cancer, ovarian cancer, prostate cancer, liver cancer, gallbladder cancer, gastrointestinal stromal tumors, biliary tract cancer, cholangiocarcinoma, acute pain, chronic pain, and polycystic kidney disease.

In other embodiments, the diseases or conditions treatable with the compounds of the present disclosure include, but are not limited to, ischemic stroke, cerebrovascular ischemia, multi-infarct dementia, head injury, spinal cord injury, Alzheimer's disease, Parkinson's disease, amyotrophic lateral sclerosis, dementia, senile chorea, Huntington's disease, neoplastic disease, complications with neoplastic disease, chemotherapy-induced hypoxia, gastrointestinal stromal tumors, prostate tumors, mast cell tumors, canine mast cell tumors, acute myeloid leukemia, acute lymphocytic leukemia, chronic myeloid leukemia, chronic lymphocytic leukemia, multiple myeloma, melanoma, mastocytosis, glioma, glioblastoma, astrocytoma, neuroblastoma, sarcomas, sarcomas of neuroectodermal origin, leiomyosarcoma, lung carcinoma, breast carcinoma, pancreatic carcinoma, colon carcinoma, hepatocellular carcinoma, renal carcinoma, carcinoma of the female genital tract, squamous cell carcinoma, carcinoma in situ, lymphoma, histiocytic lymphoma, non-Hodgkin's lymphoma, MEN2 syndromes, neurofibromatosis, Schwann cell neoplasia, myelodysplastic syndrome, leukemia, tumor angiogenesis, thyroid cancer, liver cancer, bone cancer, skin cancer, brain cancer, cancer of the central nervous system, pancreatic cancer, lung cancer, small cell lung cancer, non small cell lung cancer, breast cancer, colon cancer, bladder cancer, prostate cancer, gastrointestinal tract cancer, cancer of the endometrium, fallopian tube cancer, testicular cancer, ovarian cancer, pain of neuropathic origin, pain of inflammatory origin, acute pain, chronic pain, migraine, cardiovascular disease, heart failure, cardiac hypertrophy, thrombosis, thrombotic microangiopathy syndromes, atherosclerosis, reperfusion injury, ischemia, cerebrovascular ischemia, liver ischemia, inflammation, polycystic kidney disease, age-related macular degeneration, rheumatoid arthritis, allergic rhinitis, inflammatory bowel disease, ulcerative colitis, Crohn's disease, systemic lupus erythematosis, Sjogren's Syndrome, Wegener's granulomatosis, psoriasis, scleroderma, chronic thyroiditis, Grave's disease, myasthenia gravis, multiple sclerosis, osteoarthritis, endometriosis, dermal scarring, tissue scarring, vascular restenosis, fibrotic disorders, hypereosinophilia, CNS inflammation, pancreatitis, nephritis, atopic dermatitis, hepatitis, immunodeficiency diseases, severe combined immunodeficiency, organ transplant rejection, graft versus host disease, renal disease, prostatic disease, diabetic nephropathy, nephrosclerosis, glomerulonephritis, interstitial nephritis, Lupus nephritis, prostate hyperplasia, chronic renal failure, tubular necrosis, diabetes-associated renal complication, associated renal hypertrophy, type 1 diabetes, type 2 diabetes, metabolic syndrome, obesity, hepatic steatosis, insulin resistance, hyperglycemia, lipolysis obesity, infection, Helicobacter pylori infection, Influenza virus infection, fever, sepsis, pulmonary diseases, chronic obstructive pulmonary disease, acute respiratory distress syndrome, asthma, allergy, bronchitis, emphysema, pulmonary fibrosis, genetic developmental diseases, Noonan's syndrome, Crouzon syndrome, acrocephalo-syndactyly type I, Pfeiffer's syndrome, Jackson-Weiss syndrome, Costello syndrome, faciocutaneoskeletal syndrome, leopard syndrome, cardio-faciocutaneous syndrome, neural crest syndrome abnormalities causing cardiovascular, skeletal, intestinal, skin, hair or endocrine diseases, disorders of bone structure or mineralization, osteoporosis, increased risk of fracture, hypercalcemia, bone metastases, Grave's disease, Hirschsprung's disease, lymphoedema, selective T-cell defect, X-linked agammaglobulinemia, diabetic retinopathy, alopecia, erectile dysfunction, and tuberous sclerosis.

In some embodiments, the disease is selected from the group consisting of mast cell tumors, small cell lung cancer, testicular cancer, gastrointestinal stromal tumors (GISTs), metastatic GISTs, glioblastoma, astrocytoma, neuroblastoma, carcinomas of the female genital tract, sarcomas of neuroectodermal origin, colorectal carcinoma, carcinoma in situ, Schwann cell neoplasia associated with neurofibromatosis, acute myelocytic leukemia, acute lymphocytic leukemia, chronic myelogenous leukemia, mastocytosis, urticaria pigmentosa (UP), telangiectasia macularis eruptiva perstans (TMEP), systemic mastocytosis, indolent systemic, smoldering systemic, aggressive systemic, mast cell leukemia, mast cell sarcoma melanoma, and canine mast cell tumors, and inflammatory diseases, including asthma, rheumatoid arthritis, allergic rhinitis, multiple sclerosis, inflammatory bowel syndrome, transplant rejection, and hypereosinophilia. In certain instances, the disease is a c-kit and or c-kit mutant, such as D816F, D816H, D816N, D816Y, D816V, K642E, Y823D, Del 550-558, Del 557-561, N822K, V654A, N822H, Del 550-558+V654A, Del 557-561+V654A, Ins503AY, V560G, 558NP, Del 557-558, Del W559-560, F522C, Del 579, R634W, K642E, T801I, C809G, D820Y, N822K, N822H, Y823D, Y823C or T670I mutant-mediated disease. In one embodiment, the disease is a D816 (such as D816F, D816H, D816N, D816Y or D816V) mutant mediated disease. In another embodiment, the disease is a D816V mutant mediated disease. In yet another embodiment, the disease is a V560G mutant mediated disease. In another embodiment, the disease is a D816V and V560G mutant mediated disease. In one embodiment, the disease is a cancer, preferably selected from the group consisting of melanoma, glioma, glioblastoma multiforme, pilocytic astrocytoma, colorectal cancer, thyroid cancer, lung cancer, ovarian cancer, prostate cancer, liver cancer, gallbladder cancer, gastrointestinal stromal tumors, biliary tract cancer, and cholangiocarcinoma. In one embodiment, the cancer is melanoma, colorectal cancer, thyroid cancer or lung cancer.

In some embodiments, the disclosure provides a method for treating a disease or condition selected from urticaria pigmentosa (UP), telangiectasia macularis eruptiva perstans (TMEP), systemic mastocytosis, indolent systemic, smoldering systemic, aggressive systemic, mast cell leukemia, mast cell sarcoma, GISTs and metastatic GISTs. The method involves orally administering to the subject in need thereof an effective amount of a tablet as described herein.

In some embodiments, the disclosure provides methods for treating any c-kit protein kinase mediated disease or condition, including any c-kit mutant kinase mediated disease or condition in an animal subject in need thereof, wherein the method involves administering to the subject an effective amount of any one or more compound(s) as described herein. In certain embodiments, the method involves orally administering to the subject an effective amount of a tablet as described herein in combination with one or more other therapies for the disease or condition.

In some embodiments, the disclosure provides methods for treating any c-kit D816F, D816H, D816N, D816Y, D816V, K642E, Y823D, Del 550-558, Del 557-561, N822K, V654A, N822H, Del 550-558+V654A, Del 557-561+V654A, Ins503AY, V560G, 558NP, Del 557-558, Del W559-560, F522C, Del 579, R634W, K642E, T801I, C809G, D820Y, N822K, N822H, Y823D, Y823C or T670I mutant protein kinase mediated disease or condition in an animal subject in need thereof, wherein the method involves administering to the subject an effective amount of a tablet as described herein. In certain embodiments, the method involves administering to the subject an effective amount of a tablet as described herein in combination with one or more other therapies for the disease or condition. In some embodiments, the c-kit mutant protein kinase is c-kit D816 (such as D816F, D816H, D816N, D816Y or D816V) mutant kinase. In one embodiment, the c-kit mutant protein kinase is c-kit D816V mutant. In another embodiment, the c-kit mutant protein kinase is c-kit V560G mutant. In another embodiment, the c-kit mutant protein kinase is c-kit D816V/V560G mutant.

In some embodiments, a tablet comprising Compound (I) as described herein is a c-kit and/or mutant c-kit kinase inhibitor and has an IC₅₀ of less than 500 nM, less than 100 nM, less than 50 nM, less than 20 nM, less than 10 nM, less than 5 nM, or less than 1 nM as determined in a generally accepted c-kit kinase activity assay. In some embodiments, the compound as described herein will have an IC₅₀ of less than 500 nM, less than 100 nM, less than 50 nM, less than 20 nM, less than 10 nM, less than 5 nM, or less than 1 nM with respect to c-kit, c-kit D816V mutant, c-kit V560G mutant or D816V/V560G mutant. In some embodiments, the compound as described herein will selectively inhibit one or more mutant c-kit kinases relative to one or more other mutant c-kit kinases.

In some embodiments, the disclosure provides a method for inhibiting a c-kit mutant protein kinase, such as D816V, V560G or D816V/V560G mutant protein kinase. The method includes contacting the tablet comprising Compound (I) as described herein, with a cell or a c-kit mutant protein kinase either in vitro or in vivo.

In certain embodiments, the disclosure provides manufacture of a medicament for the treatment of a disease or condition as described herein. In other embodiments, the tablet as described herein for use in treating a disease or condition as described herein.

In specific aspects, the disease or condition treated by oral administration of the tablet of the disclosure is Acute Myeloid Leukemia (AML).

In specific aspects, the disease or condition treated by oral administration of the tablet of the disclosure is Gastrointestinal Stromal Tumors (GIST).

In specific aspects, the disease or condition treated by oral administration of the tablet of the disclosure is mastocytosis.

In specific aspects, the disease or condition treated by oral administration of the tablet of the disclosure is Advanced Systemic Mastocytosis (AdvSM).

In specific aspects, the disease or condition treated by oral administration of the tablet of the disclosure is Nonadvanced Systemic Mastocytosis (NonAdvSM).

In specific aspects, the disease or condition treated by oral administration of the tablet of the disclosure is Indolent Systemic Mastocytosis (ISM) and Smoldering Systemic Mastocytosis (SSM).

### EXAMPLES

The following examples are provided for illustrative purposes only and not to limit the scope of the claims provided herein.

### Example 1. Properties of Bezuclastinib

The chemical and physical properties of Bezuclastinib is provided in Table 1. Bezuclastinib has a high melting point and is insoluble in aqueous and most organic solvents. No suitable salt or co-crystal was found.

TABLE 1
Properties of Bezuclastinib.

| Property | Description |
|---|---|
| Molecular Weight | Free Base: 331.38 Daltons |
| Melting Point | >365° C. |
| Polymorph | Crystalline |
| Pka | 2.86 (Weak Base) |
| LogD | 3.46 |
| Aqueous solubility | 0.31 μg/mL at pH = 7.0 |

### Example 2. Solubility Studies

Solubility studies were conducted during the development of formulations comprising the active pharmaceutical ingredient (API), Bezuclastinib. The study was conducted in IV fluids, organic solvents, aqueous systems and biorelevant media. Studies were also conducted with a spray dried dispersion (SDD).

### 2.1 Solubility in Biorelevant Media

The solubility of the bezuclastinib was investigated in biorelevant media at 37° C. Biorelevant media included 0.01N hydrochloric acid, Fast State Simulated Intestinal Fluid (FaSSIF) with and without bile salts, Simulated Intestinal Fluid (SIF) with various concentrations of bile salts (0, 0.5% and 1%) and Fasted State Simulated Gastric Fluid (FaSSGF). A saturated suspension was prepared in each media and the samples were stirred overnight using a magnetic stirrer. The free drug concentration was determined by subjecting the samples to 7 minutes in an ultracentrifuge at 470,000× gravity. A 100 μL aliquot was withdrawn and diluted in 5× in DMSO and Methanol and tested by the HPLC method described in Table 2. Six (6) replicates were performed in each media. In all cases, bezuclastinib was practically insoluble (free drug concentration ≤1.2 μg/mL).

TABLE 2
HPLC Method.

| Parameter | Description | |
|---|---|---|
| Column | Waters XBridge BEH C18, 3.5 μ, | |
| | 4.6 mm × 50 mm, PN: 186003031, | |
| | or equivalent | |
| Mobile Phase A | 0.1% Formic Acid in Water | |
| Mobile Phase B | 0.1% Formic Acid in Acetonitrile | |
| Standard Diluent | 70:30 Acetonitrile:DMSO | |
| Sample Diluent | 2.0% CTAB in 50 mM NaH2PO4, | |
| | pH 6.8 | |
| Run Time (mn) | 8 | |
| Flow Rate | 1.5 | mL/min |
| Column Temperature | 55° | C. |
| Injection Volume | 10 | μL |
| Detection Wavelength | 332 | nm |

| Gradient Program | Time (min) | % MP A | % MP B |
|---|---|---|---|
| | 0 | 80 | 20 |
| | 1 | 80 | 20 |
| | 5 | 0 | 100 |
| | 6 | 0 | 100 |
| | 6.1 | 80 | 20 |
| | 8 | 80 | 20 |

### 2.2 Solubility in Organic Solvents

The solubility of bezuclastinib was investigated in organic solvents at 21° C. (Table 3). A saturated suspension was prepared in each solvent and the samples were stirred overnight using a magnetic stirrer. The free drug concentration was determined by subjecting the samples to 7 minutes in an ultracentrifuge at 470,000× gravity. A 100 μL aliquot was withdrawn and diluted in 5× in DMSO and Methanol and tested by the HPLC method. Three (3) replicates were performed in each solvent. Bezuclastinib was poorly soluble (free drug concentration <5.0 mg/mL), other than DMSO (free drug concentration <20 mg/mL).

TABLE 3
Organic Solvents used to Assay Solubility of API.
Media
Acetone
Acetic Acid
Acetonitrile
DCM
Diethylether
DMF
DMSO
Ethyl Acetate
Isopropyl Alcohol
Methanol
2,2,2-trifluoroethanol
Water
Ethanol
85/15 THF/water
Dimethylacetamide
NMP
Formic Acid
2-methyl THF

### 2.3 Influence of pH on Solubility

The influence of pH on the solubility of the bezuclastinib was investigated in Simulated Intestinal Fluid with 0.5% bile salts at 37° C. at pH 4.0, 5.5 and 6.0. A saturated bezuclastinib suspension was prepared in each media and the samples were stirred overnight using a magnetic stirrer. The total drug concentration was determined by subjecting the samples to 3 minutes in a microcentrifuge at 19,500× gravity. The free drug concentration was determined by subjecting the samples to 7 minutes in an ultracentrifuge at 470,000× gravity. A 100 μL aliquot was withdrawn and diluted in 5× in DMSO and Methanol and tested by the HPLC method. Six (6) replicates were performed in each media. In all conditions, bezuclastinib was poorly soluble (free drug concentration <20 μg/mL).

### Example 3. Amorphous Solid Dispersions Formulation Studies

Due to the poor water solubility and lipophilicity of bezuclastinib, amorphous dispersion techniques were investigated to improve the bioavailability.

In an ASD, the solubility of the drug substance is improved by disarranging its crystalline lattice to produce a higher energy state of amorphous form. Polymers also play a key role to improve the solubility and bioavailability of amorphous API by drug polymer interaction. As shown below in this Example 3, numerous amorphous solid dispersion techniques were tried to solve the known low solubility in lipophilicity of bezuclastinib. Applicant surprisingly discovered that a particular polymer, and even grade of polymer, yielded unexpectedly superior results. The polymer could stabilize the ASD and prevent the drug from crystallization and to provide improved physical stability under a variety of accelerated stability conditions, such as elevated temperature and relative humidity. Under this approach, the following studies were conducted.

### 3.1. KinetiSol®

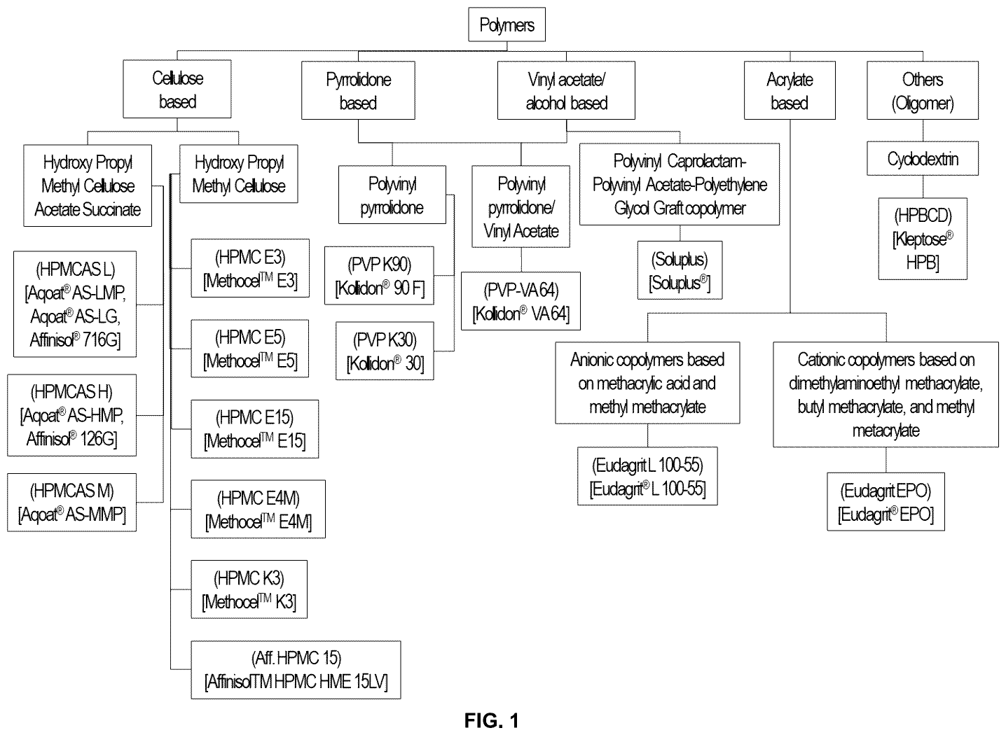

KinetiSol® is a fusion-based process for the manufacture of amorphous solid dispersion systems (“KSD”), Ellenberger et al., (AAPS PharmSciTech, 2018), incorporated herein by reference. The KinetiSol® approach was applied to the API in an effort to generate the best amorphous solid dispersion. The initial polymer screening with the API using the KinetiSol® technology was performed, as shown in FIG. 1. It was found that hypromellose (HPMC) and hypromellose acetate succinate (HPMCAS) polymers were promising, although several polymers appeared to be performing well in vitro.

Bezuclastinib KSDs were prepared using a KinetiSol® small-scale compounder (Formulator II) and large-scale compounders (Batch Compounder, GMP Gen 1 Continuous/Batch Compounder) designed and manufactured by DisperSol Technologies LLC (Georgetown, TX, USA). Before compounding, the API and polymer/oligomer excipients were accurately weighed and blended to prepare physical mixtures (PMs). These physical mixtures were charged into the KinetiSol® compounder chamber. Inside the chamber, a shaft with protruding blades was rotated at varying incremental speeds without the addition of external heat in order to impart frictional and shear forces to the sample material. The temperature of the mass was monitored using an infrared probe. When the molten mass temperature reached the target temperature, the mass was rapidly ejected, collected, and pressed between two stainless steel plates to rapidly quench the sample. Set stages ranged from 3,000 to 7,2000 rpm and set temperatures ranging from 180° C. to 250° C.

The quenched mass obtained after KinetiSol® processing was milled using a lab scale rotor mill (i.e., IKA tube mill 100 (IKA Works GmbH & Co. KG, Staufen, Germany)). For milling, the fragments of quenched mass were loaded into a 20 mL grinding chamber, which was operated for 30-60 s with a grinding speed between 10,000 and 20,000 rpm. This milled material was subsequently passed through a #60 mesh screen (<250 μm). Material retained above the screen (i.e., >250 μm) was cycled through the mill with the same parameters. This process of milling and sieving was repeated until all material passed through the screen. The resultant material (<250 μm) was labeled as KSD.

Four batches of formulations (Polymer (% w/w): HPMCAS-LMP (85); HPMCAS-LMP (80); HPMCAS-LMP (5); and HPMC E5 (68)) were selected to complete the stability study. The formulations were found to be both physically and chemically stable for four weeks at accelerated conditions when in closed packaging. Animal studies performed in rats and dogs demonstrated that the HPMCAS E5 and HPMCAS LMP based formulations provided increased bioavailability compared to the other formulations, including the spray dried solid dispersion used as a control. However, low assay and total impurities of greater than 2% were formed during processing. In addition, the appearance of the dispersion was dark indicating possible polymer degradation was observed. Thus, the KinetiSol® approach was abandoned.

### 3.2. Micro Precipitated Bulk Powder (“MBP”)

MBP is a solvent-controlled coprecipitation process in which the API was dissolved in DMSO with a polymer followed by precipitation in an antisolvent (acidified, cold water) with the goal to yield a polymer-stabilized amorphous-solid dispersion. Methods of preparing samples for this MBP process can be found in Shah, et al. (Journal of Pharmaceutical Sciences, Vol. 102, No. 3, 2013), and in U.S. Pat. No. 9,447,089 B2, incorporated herein by reference.

MBP samples were prepared at 20% and 15% drug loading using 2 grades of HPMCAS polymer, as shown in Table 4. The API polymer solution to anti-solvent ratio was 1:10, the anti-solvent pH was 2.5 and the anti-solvent temperature was 2-5° C. The precipitate was collected and washed with chilled anti-solvent one time followed by chilled purified water, then dried at 30° C. X-ray powder diffraction (XRPD) technique was used to analyze the MPB samples.

Crystalline peaks associated with the API were present in each of the MBP samples. After storage at 40° C. and 75% relative humidity for 1 week, the intensity of the crystalline peaks in the MBP samples increased under these storage conditions. Thus, the MBP approach was abandoned because fully amorphous dispersions could not be achieved.

TABLE 4
Formulations for MBP Approach.

| | Formulation # | | | |
|---|---|---|---|---|
| Component | 1 | 2 | 3 | 4 |
| API | 20 | 20 | 15 | 15 |
| HPMCAS L | 80 | | 85 | |
| HPMCAS H | | 80 | | 85 |

### 3.3. Hot Process Spray Drying

The spray drying process is disclosed in WO 2010/111132 A2, incorporated herein by reference. A “hot process” spray drying technique in which the feed suspension is fed through a heat exchanger to dissolve drug in pressurized lines in order to raise active solubility prior to entering the drying chamber was used in this study.

The API-SDD prototype manufacture involved the following polymers and drug loadings for this study includes 10% drug-loading HPMCAS-L, —H; 20% drug-loading HPMCAS-H; 30% drug-loading HPMCAS-L, —H; 30% drug-loading PVPVA64; and 20% drug loading HPMCP.

All SDDs from this hot process exhibited partial crystallinity by XRPD. The chemical stability of the hot process SDDs was assessed by HPLC. Potency was off target for most SDDs due to manufacturing issues in which the API precipitated and was captured in the inline filter. Low levels of impurities were formed during hot process spray drying (<0.16). Due to the in process precipitation manufacturing issues, hot process spray drying was abandoned.

### 3.4. Spray Drying

Screening methodologies for the development of spray-dried amorphous solid dispersions has been assessed in Duarte, et al., Pharm Res (2015) 32:222-237, the entire contents of which are incorporated by reference herein. Spray-dried dispersions (SDDs) of low-solubility drugs prepared using the polymer hydroxypropyl methylcellulose acetate succinate (HPMCAS) have been described in Friesen, et al., Molecular Pharmaceutics, 2008, Vol. 5, No. 6, 1003-1019, the entire contents of which are incorporated by reference herein. The interplay between manufacturing process, formulation parameters, physical structure, and performance of the solid dispersions with respect to stability and drug release characteristics have been studied in Paudel, et al., Int. J. Pharmaceutics, 2013, 453, 253-284, the entire contents of which are incorporated by reference herein. The level of sophistication that can be achieved in the area of particle engineering via spray drying is described in Vehring, Pharmaceutical Research, 2008, Vol. 25, the entire contents of which are incorporated by reference herein.

Method for making homogenous spray-dried solid amorphous dispersions of drugs using pressure nozzles is disclosed in U.S. Pat. No. 7,780,988 B2, the entire contents of which are incorporated by reference herein.

Methods and compositions to improve bioavailability of active agents and achieve rapid dissolution of drug from spray-dried dispersions in capsules is disclosed in US 2018/0161269A1, the entire contents of which are incorporated by reference herein.

A spray drying process characterized by continuous preparation and immediate spray drying of a solution comprising API, excipient and solvent is disclosed in WO 2019/162688A1, the entire contents of which are incorporated by reference herein.

U.S. Pat. No. 8,216,495B2 disclose preparation of poorly soluble drugs in a solid dispersion by spray-drying, the entire contents of which are incorporated by reference herein.

A spray drying process for forming pharmaceutical compositions comprising a solid amorphous dispersion of a low-solubility drug and a polymer is disclosed in US 2005/0031692A1, the entire contents of which are incorporated by reference herein.

Spray Dried Dispersions (SDDs) were prepared using THF and water solvent system due to the limited solubility of the API in volatile organic solvents.

The API-SDD prototype manufacture involved the following polymers and drug loadings for this study includes 10% drug-loading HPMCAS-L, -H; 15% drug-loading HPMCAS-H; 20% drug-loading HPMCAS-L, -M, -H; 20% drug-loading CAP; 20% drug-loading EudagritL100; and 20% drug loading HPMCP. The SDD samples were examined by XRPD from 3-40 2θ at a scan rate of 2° per minute. All SDD appeared amorphous by XRPD for the 20% drug loaded compositions.

### 3.4.1. Non-Sink Dissolution Test

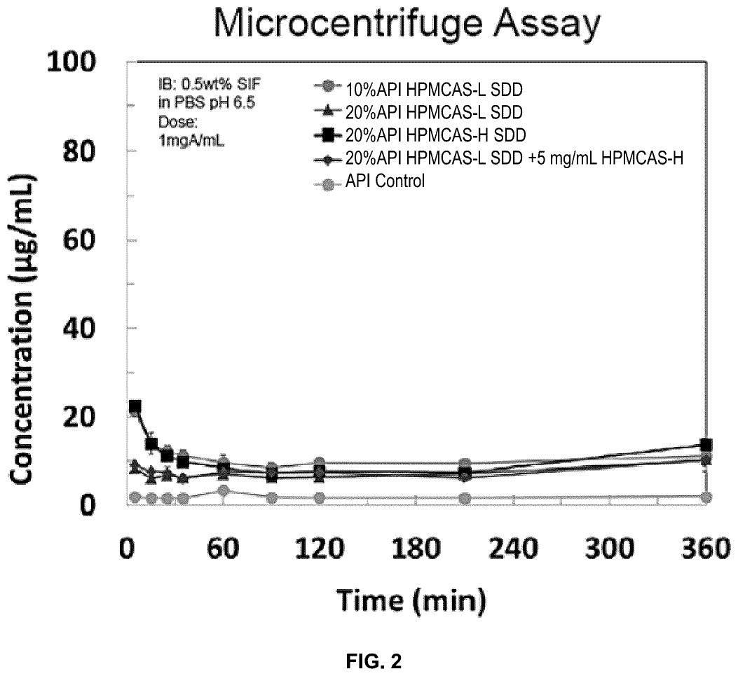

Non-sink dissolution testing was used to determine the kinetic solubility as well as the extent and duration of supersaturation of amorphous dispersions. Spray dried samples were examined under non sink dissolution testing conditions in FIGS. 2-5. Non sink testing was performed with and without a gastric transfer at various bile salt concentrations in biorelevant media including simulated intestinal fluid at pH 6.5 and FaSSIF at pH 6.5. Total drug and free drug concentration were determined as described in Example 2.

FIGS. 2-5 show the assays from the non-sink dissolution test. In general, HPMCAS performed best on non-sink dissolution testing. Grade H sustained free drug to a greater extent than the other polymers. Results also indicate that 10% drug loading outperformed 20% drug loading. In terms of free drug sustainment, 20% HPMCAS-H SDD performed better than the 10% HPMCAS-L SDD. Physical mixing of HPMCAS-H to 20% HPMCAS-L SDD had no impact.

FIG. 2 shows the non-sink total drug dissolution testing results of SDD samples compared to crystalline API in simulated intestinal fluid with 0.5% bile salt concentration at a pH of 6.5 and 37° C. Samples were loaded at a concentration of 1 mg API per mL of media. The SDD's tested were 10% and 20% drug loading HPMCAS-L SDD, 20% HPMCAS-H and 20% HPMCAS-L with 5 mg/mL HPMCAS-H added.

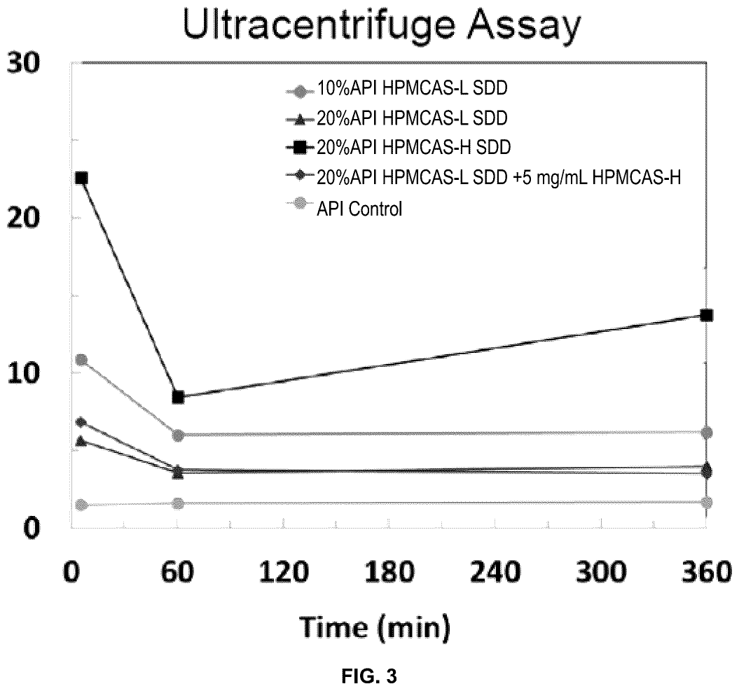

FIG. 3 shows the non-sink free drug dissolution testing results of SDD samples compared to crystalline API in simulated intestinal fluid with 0.5% bile salt concentration at a pH of 6.5 and 37° C. Samples were loaded at a concentration of 1 mg API per mL of media. The SDD's tested were 10% and 20% drug loading HPMCAS-L SDD, 20% HPMCAS-H and 20% HPMCAS-L with 5 mg/mL HPMCAS-H added.

The total drug (microcentrifuge assay) and free drug (ultracentrifuge assay) concentrations are presented in Table 5.

TABLE 5
Formulations for Microcentrifuge and Ultracentrifuge Assay.

| | Micro- | Ultra- | Micro- | Ultra- | Micro- | Ultra- | | |
|---|---|---|---|---|---|---|---|---|
| | centrifuge | centrifuge | centrifuge | centrifuge | centrifuge | centrifuge | | |
| | Assay @ | Assay @ | Assay @ | Assay @ | Assay @ | Assay @ | Micro | Ultra |
| | 5 min | 5 min | 60 min | 60 min | 6 hrs | 6 hrs | AUC₃₆₀ | AUC₃₆₀ |
| Formulation | (μg/mL) | (μg/mL) | (μg/mL) | (μg/mL) | (μg/mL) | (μg/mL) | (ug*min⁻¹*mL⁻¹) | (ug*min⁻¹*mL⁻¹) |
| 10% API 90% | 21 | 11 | 10 | 6.0 | 11 | 6.2 | 3690 | 2320 |
| HPMCAS-L | | | | | | | | |
| 20% API 80% | 8.4 | 5.8 | 7.1 | 3.5 | 10 | 4.0 | 2680 | 1400 |
| HPMCAS-L | | | | | | | | |
| 20% API 80% | 23 | 20 | 8.4 | 5.3 | 14 | 8.9 | 3410 | 2870 |
| HPMCAS-H | | | | | | | | |
| 20% API 80% | 9.1 | 6.9 | 7.5 | 3.8 | 10 | 3.5 | 2720 | 1400 |
| (HPMCAS-L + | | | | | | | | |
| HPMCAS-H) | | | | | | | | |
| API | 2.0 | 1.5 | 3.4 | 1.6 | 2.0 | 1.7 | 670 | 590 |

All the SDD's outperformed the crystalline API in terms of total and free drug. Total and free drug concentration was higher through 60 minutes with the 10% drug loading in HPMCAS-L than the 20% loaded SDD's. However, at the 6-hour time point, 20% HPMCAS-H produced the highest total and free drug concentrations. 20% HPMCAS-H also produced the highest area under the curve for free drug concentration while 10% HPMCAS-L produced the highest area under the curve for total drug concentration.

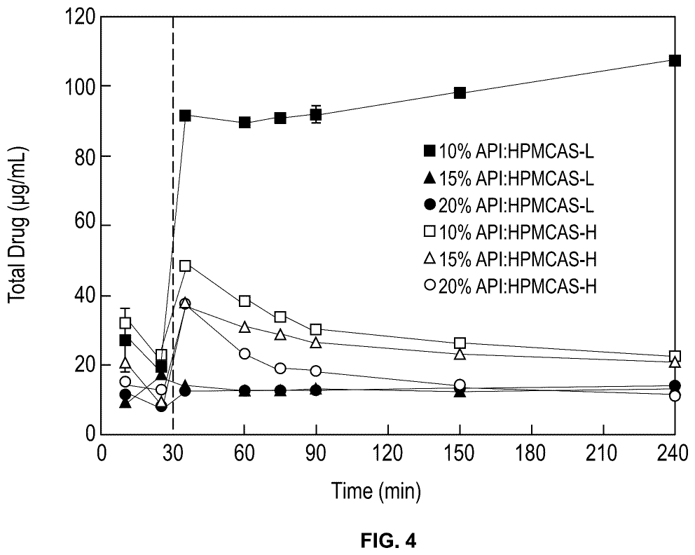

FIG. 4 shows the non-sink, total drug dissolution testing results of 10%, 15% and 20% drug loaded SDD samples with a gastric transfer at 37° C. Samples were loaded at a concentration of 3 mg API per mL in 0.01 N hydrochloric acid (pH 2) for 30 minutes, then diluted to 1 mg API per mL in simulated intestinal fluid with 0.224% bile salts at pH 6.5. The total drug concentrations at the 60-minute time point are presented in Table 6.

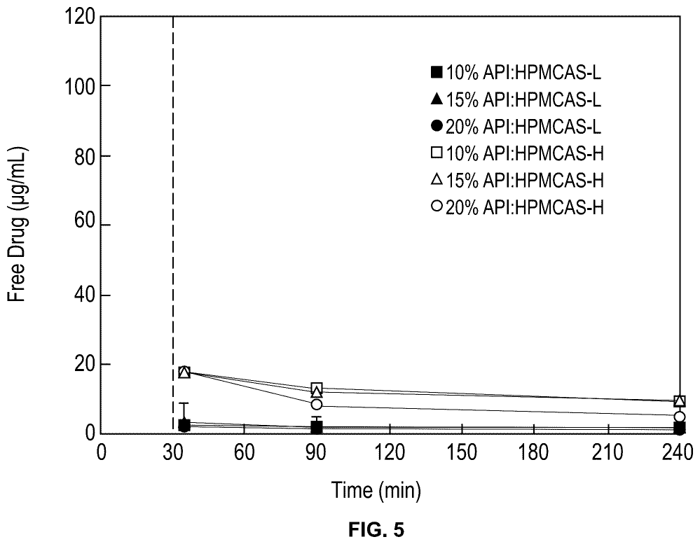

FIG. 5 shows the non-sink, free drug dissolution testing results of 10%, 15% and 20% drug loaded SDD samples with a gastric transfer at 37° C. The free drug concentrations and the total concentration at the 60-minute time point is presented in Table 6.

TABLE 6
SDD Formulations for Microcentrifuge and Ultracentrifuge Assay.

| | Microcentrifuge | Ultracentrifuge | Microcentrifuge | Ultracentrifuge | Microcentrifuge | Ultracentrifuge |
|---|---|---|---|---|---|---|
| | Assay @ | Assay @ | Assay @ | Assay @ | Assay @ | Assay @ |
| | 5 min | 5 min | 60 min | 60 min | 210 min | 210 min |
| Formulation | (μg/mL) | (μg/mL) | (μg/mL) | (μg/mL) | (μg/mL) | (μg/mL) |
| 10% API 90% | 91 | 2.3 | 92 | 1.8 | 107 | 1.6 |
| HPMCAS-L | | | | | | |
| 15% API 85% | 13 | 2.1 | 12 | 1.4 | 13 | 1.2 |
| HPMCAS-L | | | | | | |
| 20% API 80% | 12 | 1.7 | 13 | 1.2 | 14 | 1.1 |
| HPMCAS-L | | | | | | |
| 10% API 90% | 48 | 7 | 30 | 3 | 22 | 9.1 |
| HPMCAS-H | | | | | | |
| 15% API 85% | 37 | 18 | 26 | 12 | 20 | 9.5 |
| HPMCAS-H | | | | | | |
| 20% API 80% | 37 | 18 | 18 | 8.2 | 11 | 5.2 |
| HPMCAS-H | | | | | | |

Results from Table 6 demonstrate that all HPMCAS-H SDDs show significant improvement in free drug and sustainment versus HPMCAS-L SDDs. The 15% API-85% HPMCAS-H SDDs appeared to perform as well as the 10% API-90% HPMCAS-H SDDs, and both are better than the 20% API-80% HPMCAS-H SDDs.

The 10% API-90% HPMCAS-L SDDs formed colloids, and the free drug values suggest that precipitation has occurred. The 15% and 20% API-HPMCAS-L SDDs formed very few colloids and have poor sustainment of supersaturation.

### 3.4.2. 4-Week Stability Test

The SDDs samples were placed on stability at 40° C. and 75% relative humidity in open containers and at 40° C. under ambient humidity in closed containers to assess their physical stability by XRPD and their glass transition temperature by Differential Scanning Calorimetry (DSC). The chemical stability of the SDDs was assessed by HPLC.

The HPMCAS-H and HMPCAS-L SDD samples were analyzed by XRPD to confirm they were amorphous prior to the stability study. After 4 weeks exposure to these conditions in closed containers, very slight crystallinity was observed in 20% API 80% HPMCAS-L formulation. All other SDDs look to not have crystals observed by XRPD.

After 4 weeks exposure to these conditions in open containers, the samples with 15% and 20% drug loading were found to have crystal formation. Both the SDDS with 10% drug loading appeared to not have crystals present. The 20% API 80% HPMCAS-L SDDs look to have more significant crystallization than the 20% API 80% HPMCAS-H SDDs.

The glass transition temperature was measured using Modulated Differential Scanning Calorimetry (Ramp from 25-375° C. at 3° C./min modulated at 1° C./min). mDSC analysis of the SDDs from the stability study was performed. All formulations demonstrated a change in glass transition temperature indicating physical changes had occurred under these conditions. The chemical stability and impurities of the SDDs was analyzed by HPLC. Minimal impurity growth was observed after 4 weeks at stability conditions in HPMCAS-H lots. More impurity growth was observed in the HPMCAS-L lots compared to HPMCAS-H lots, and there was an increase in impurities with increased drug loading.

### Example 4. Evaluation of SDD and KSD Amorphous Solid Dispersions (ASD) in Rat

The SDD and KSD amorphous solid dispersions (ASD) were evaluated in a single-dose pharmacokinetic study in male Sprague Dawley Rats as described below. Six rats were administered the amorphous dispersion by oral gavage in 0.5% methylcellulose (Methocel A4M) in water at a dose of 100 mg/kg. The rats were 8 to 14 weeks of age at the start of dosing and weighed between 275 to 325 grams. The animals were fasted overnight prior to oral dosing and then presented food ˜4 hours post-dose. Water was available ad libitum and each animal was acclimated to their laboratory environment for a minimum of one day prior to the study.

Serial blood samples (˜0.3 mL each), were obtained from each animal via the jugular vein catheter at the following timepoints: pre-dose and 2, 4, 6, 8 and 24 hours post-dose.

The Area Under the Curve (AUC) and the Cₘₐₓ for the SDD and KSD formulations are provided in Table 7. The 10% HPMCAS-L KSD produced the highest AUC and Cₘₐₓ for all the amorphous dispersions. Among the SDD, the 10% HPMCAS-H SDD produced the highest AUC and Cₘₐₓ.

TABLE 7
Summary of Rat TK Data - SDD and KSD Amorphous
Solid Dispersions.

| ASD | | Dose | AUC ₀₋₂₄ | Cₘₐₓ |
|---|---|---|---|---|
| Type | Formulation | (mg/kg) | (hr*ng/mL) | (ng/mL) |
| SDD | 10% API 90% HPMCAS-H | 100 | 31,123 | 2,772 |
| | SDD | | | |
| | 15% API 85% HPMCAS-H | 100 | 18,345 | 1,741 |
| | SDD | | | |
| | 20% API 80% HPMCAS-H | 100 | 15,253 | 1,365 |
| | SDD | | | |
| | 10% API 90% HPMCAS-L | 100 | 22,775 | 1,898 |
| | SDD | | | |
| KSD | 10% API 90% HPMCAS-L | 100 | 113,504 | 8,903 |
| | KSD | | | |
| | 20% API 34% HPMCAS-L | 100 | 35,569 | 2,122 |
| | 45% PVP KSD | | | |
| | 10% API 90% Copovidone | 100 | 9,962 | 878 |
| | KSD | | | |
| | 10% API 90% PVP KSD | 100 | 10,388 | 1,070 |
| | 10% API 90% HPMC KSD | 100 | 39,283 | 3,450 |
| | 10% API 70% HPMCAS-L | 100 | 26,939 | 2,818 |
| | 20% PVP KSD | | | |
| | 10% API 70% HPMCAS-H | 100 | 34,908 | 3,467 |
| | 20% PVP KSD | | | |

### Example 5. Development of Tablet Formulation

The tablets comprising the API were manufactured by a roller compaction/dry granulation (RCDG) process. The influence of binder properties on dry granules and tablets were studied by Arndt, et al., Powder Tech. 2018, 337, 68-77, the entire contents of which are incorporated by reference herein. The suitability of different dry binders for roll compaction/dry granulation was evaluated by Herting et al., Pharmaceutical Devel. and Tech. 2007, 12:5, 525-532, the entire contents of which are incorporated by reference herein. The effect of raw material particle size on granule and tablet properties was studied by Herting et al., International J. of Pharmaceutics 2007, 338, 110-118, the entire contents of which are incorporated by reference herein. The compaction behavior of dry granulated binary mixtures was investigated by Gandarillas et al., Powder Tech. 2015, 285, 62-67, the entire contents of which are incorporated by reference herein.

Due to the low drug loading in the SDDs, direct compression failed to produce quality tables. Wet granulation is generally not used for amorphous dispersions since it causes recrystallization.

Tablets were formulated with 2 different processes. Tablets developed from Process A yielded Formulation A. Tablets developed from Process B yielded Formulation B.

5.1. Tablet Formulation: Process a—Formulation A

Following spray drying using HPMCAS-L as the polymer, the HPMCAS-L SDD was blended with colloidal silicon dioxide and magnesium stearate. This blend was roller compacted to form granules. The granules were blended with Copovidone, Crospovidone, Croscarmellose Sodium, Mannitol, Sodium Lauryl Sulfate, Poloxamer 407, Sodium Chloride and Sodium Bicarbonate.

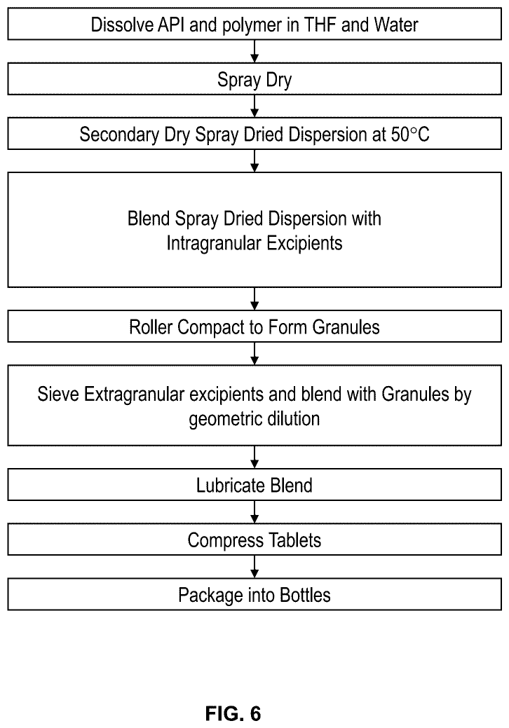

This blend was lubricated with additional magnesium stearate and compressed into tablets with a target weight of 950.1 mg and comprising 50 mg API. The uncoated tablet composition is provided in Table 8 and the process flow diagram is provided in FIG. 6.

TABLE 8
Tablet Formulation A - 50 mg dose.

| | | Percent | Amount |
|---|---|---|---|
| | Material | (w/w) | (mg/tablet) |
| SDD | API | 5.3 | 50.0 |
| | Hypromellose Acetate Succinate | 46.2 | 438.9 |
| | (HPMCAS-L) | | |
| | Butylated Hydroxytoluene (BHT) | 1.2 | 11.1 |
| Intra | Colloidal Silicon Dioxide | 1.0 | 9.5 |
| Granular | Magnesium Stearate | 0.5 | 4.8 |
| Extra | Copovidone | 8.0 | 76.0 |
| Granular | Croscarmellose Sodium | 3.0 | 28.5 |
| | Crospovidone | 6.0 | 57.0 |
| | Mannitol | 8.4 | 79.8 |
| | Sodium Lauryl Sulfate | 2.0 | 19.0 |
| | Poloxamer | 7.0 | 66.5 |
| | Sodium Chloride | 3.0 | 28.5 |
| | Sodium Bicarbonate | 8.0 | 76.0 |
| | Magnesium Stearate | 0.5 | 4.8 |
| | Total | 100.0 | 950.1 |

As discussed below, Process A and its formulation was abandoned after analysis of human pharmacokinetic data in comparison to that of the formulation obtained by Process B.

### 5.2. Tablet Formulation: Process B—Formulation B

Following spray drying using HPMCAS-H as the polymer, the HPMCAS-H SDD was blended with Microcrystalline cellulose, Mannitol, Croscarmellose Sodium, Colloidal Silicon Dioxide and Sodium Stearyl Fumarate.

This blend was roller compacted to form granules. The granules were blended with Croscarmellose Sodium, Colloidal Silicon Dioxide and Sodium Stearyl Fumarate. This blend was compressed into tablets with a target weight of 715 mg and comprising 50 mg API and tablets with a target weight of 1,072.5 mg comprising 75 mg API. The uncoated tablet compositions are provided in Table 9 and Table 10, and the process flow diagram is provided in FIG. 6.

TABLE 9
Tablet Formulation B - 50 mg dose.

| | | Percent | Amount |
|---|---|---|---|
| | Material | (w/w) | (mg/tablet) |
| SDD | API | 6.993 | 50.0 |
| | Hypromellose Acetate Succinate | 62.937 | |
| | (HPMCAS-H) | | |
| Intra | MCC | 7.91 | 56.6 |
| Granular | Mannitol | 7.91 | 56.6 |
| | Croscarmellose Sodium | 8.00 | 57.2 |
| | Colloidal Silicon Dioxide | 0.50 | 3.6 |
| | Sodium Stearyl Fumarate | 0.75 | 5.4 |
| | Croscarmellose Sodium | 4.00 | 28.6 |
| | Colloidal Silicon Dioxide | 0.50 | 3.6 |
| | Sodium Stearyl Fumarate | 0.50 | 3.6 |
| | Total | 100.0 | 715.0 |

TABLE 10
Tablet Formulation B - 75 mg dose.

| | ACTIVE | |
|---|---|---|
| | Percent | Amount |
| Material | (w/w) | (mg/tablet) |
| 10:90 API:HPMCAS - H SDD | 69.930 | 750.0 |
| MCC | 7.910 | 84.8 |
| Mannitol | 7.910 | 84.8 |
| Croscarmellose Sodium | 8.000 | 85.8 |
| Colloidal Silicon Dioxide | 0.500 | 5.4 |
| Sodium Stearyl Fumarate | 0.750 | 8.0 |
| Croscarmellose Sodium | 4.000 | 42.9 |
| Colloidal Silicon Dioxide | 0.500 | 5.4 |
| Sodium Stearyl Fumarate | 0.500 | 5.4 |
| Total | 100.0 | 1,072.5 |

### Example 6. Evaluation of Formulation B in Non Human Primates (NHP)

The SDD and KSD amorphous dispersions were formulated into tablets of Formulation B and evaluated in a single-dose pharmnacokinetic study in non-human primates (cynomolgus monkeys). Three monkeys for each composition were administered the tablets at doses of 25-50 mg/kg.

The animals were fasted 2 hours prior to oral dosing and then presented food ˜2 hours post-dose. Water was available ad libitum. Serial blood samples (˜0.5 mL each), were obtained from each animal via the femoral, saphenous, or other available vein via direct venipuncture at the following timepoints: pre-dose and 2, 4, 6, 8 and 24 hours post-dose. The results of the study are provided in Table 11.

TABLE 11
Summary of Non-Human Primate TK Data -
SDD and KSD Amorphous Dispersions.

| ASD | Formu- | Dose | Cₘₐₓ | AUCₗₐₛₜ | Cₘₐₓ/ | AUCₗₐₛₜ/ |
|---|---|---|---|---|---|---|
| Type | lation | (mg/kg) | (ng/mL) | (h*ng/mL) | dose | Dose |
| SDD | 10% | 38.12 | 1220 | 19,100 | 32.0 | 501.0 |
| | HPMCAS-H | | | | | |
| | 15% | 50 | 454 | 6,230 | 9.1 | 124.6 |
| | HPMCAS-H | | | | | |
| | 20% | 25 | 276 | 4,140 | 11.0 | 165.6 |
| | HPMCAS-H | | | | | |
| | 10% | 25 | 490 | 9,370 | 19.6 | 374.8 |
| | HPMCAS-L | | | | | |
| KSD | 10% | 50 | 1810 | 29,100 | 36.2 | 582.0 |
| | HPMCAS-L | | | | | |
| | 10% | 25 | 418 | 8,420 | 16.7 | 336.8 |
| | HPMCAS-L | | | | | |
| | (70) + PVP | | | | | |
| | K90 (20) | | | | | |
| | 10% | 25 | 598 | 9,900 | 23.9 | 396.0 |
| | HPMCAS-H | | | | | |
| | (70) PVP | | | | | |
| | K90 (20) | | | | | |

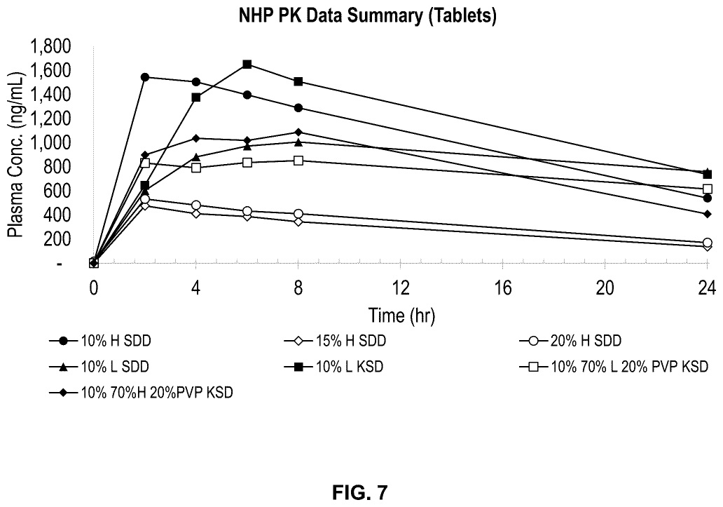

FIG. 7 shows the plasma concentration time curves of the tablets made with SDD and KSD amorphous dispersions in non-human primates.

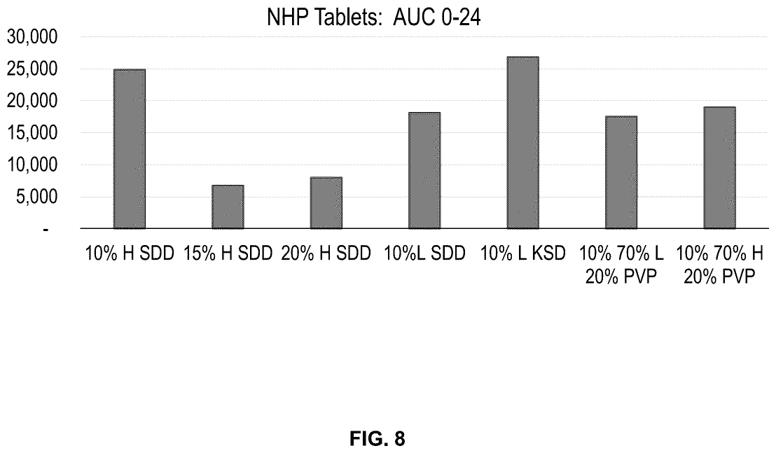

FIG. 8 shows the area under the curve of the tablets made with SDD and KSD amorphous dispersions in non-human primates.

The 10% HPMCAS-L KSD produced the highest AUC per dose and Cₘₐₓ per dose for all the amorphous dispersions. Among the SDDs, the 10% HPMCAS-H SDD produced the highest AUC and Cₘₐₓ, Lower bezuclastinib exposure was observed in the 15% and 20% drug loaded SDD compared to the 10% drug loaded SDD.

### Example 7. Evaluation of Formulations A and B in Humans

This Example summarizes a single-center, open-label, 2-period, randomized, crossover study of Formulations A and B of bezuclastinib at single ascending dose levels in healthy adult subjects to assess pharmacokinetics and relative bioavailability. Subjects were randomized to receive either a single dose of Formulation A fasted, or a single dose of Formulation B fasted. After a washout period of 21 days, subjects were then crossed over to receive the other formulation. Thirty subjects (10/cohort) received single oral doses of 50 mg (Cohort 1), 300 mg (Cohort 2) and 600 mg (Cohort 3) of Formulation A and Formulation B. Subsequently, a 1,000 mg dose of Formulation A was administered. Blood draws were taken pre-dose, and 0.5, 1, 2, 3, 4, 6, 8, 12, 16, 24, 48, 72, 96, 120, 144, 168, 192, and 336 h post-dose.

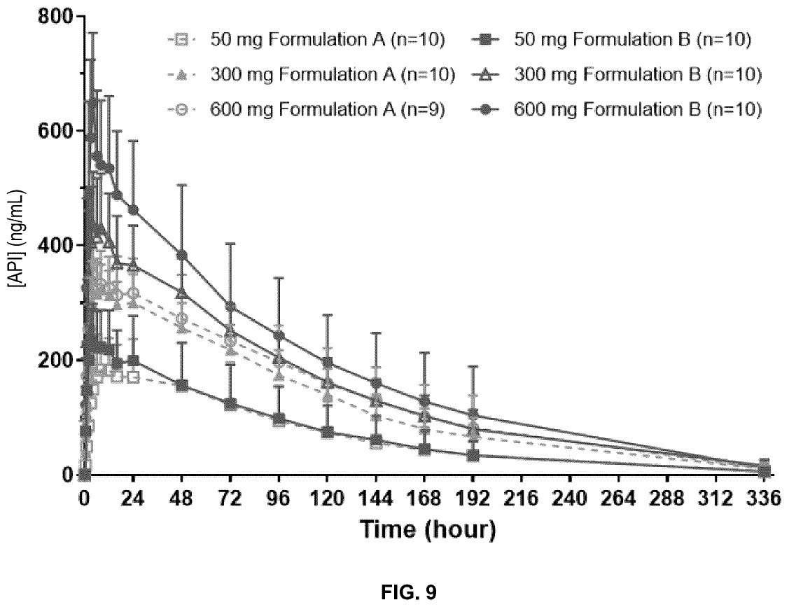

FIG. 9 shows the plasma concentration time curves of Formulations A and B through 336 hours.

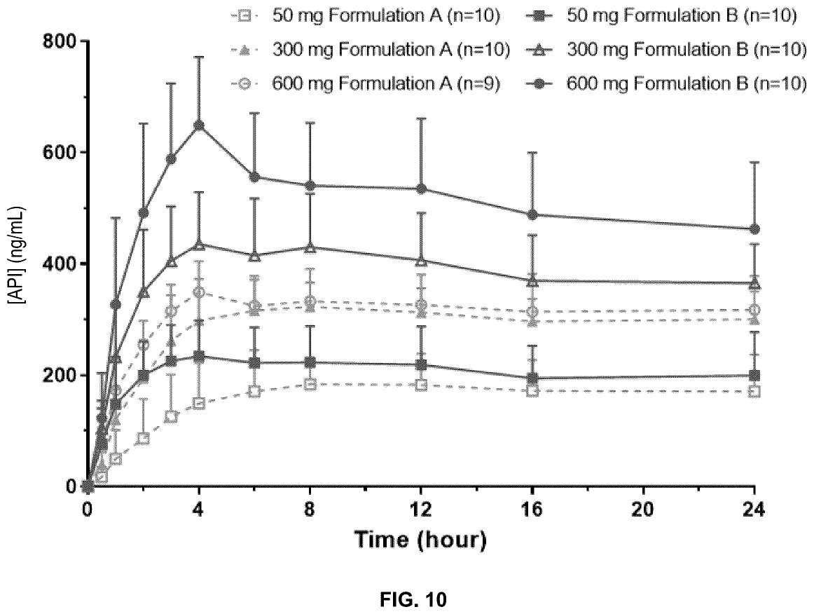

FIG. 10 shows the plasma concentration time curves of Formulations A and B through 24 hours.

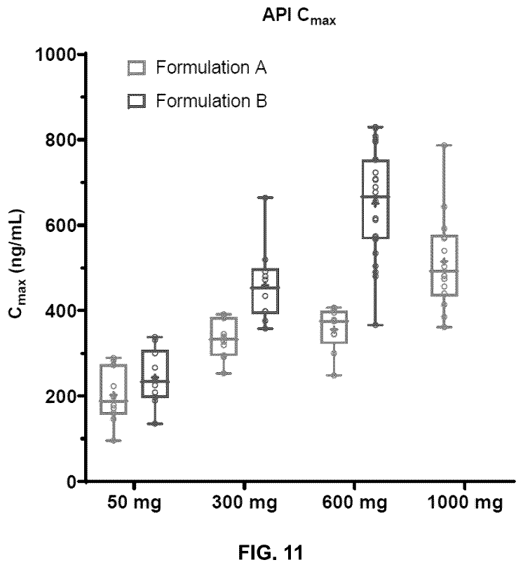

FIG. 11 compares the Cₘax of Formulations A & B.

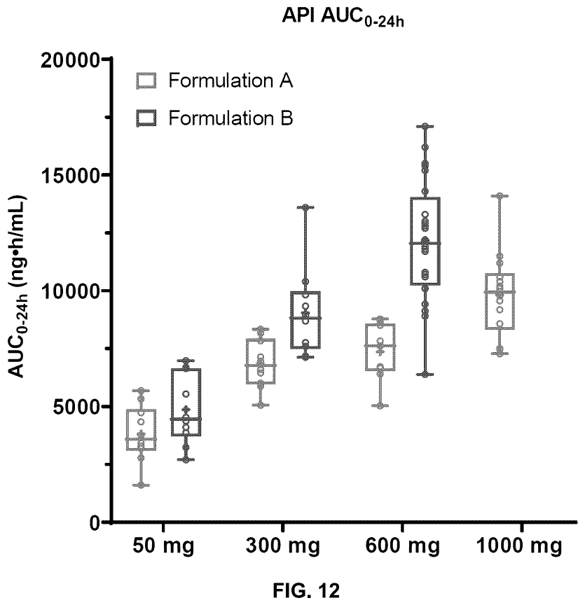

FIG. 12 compares the Area Under the Curve (AUC) of Formulations A & B.

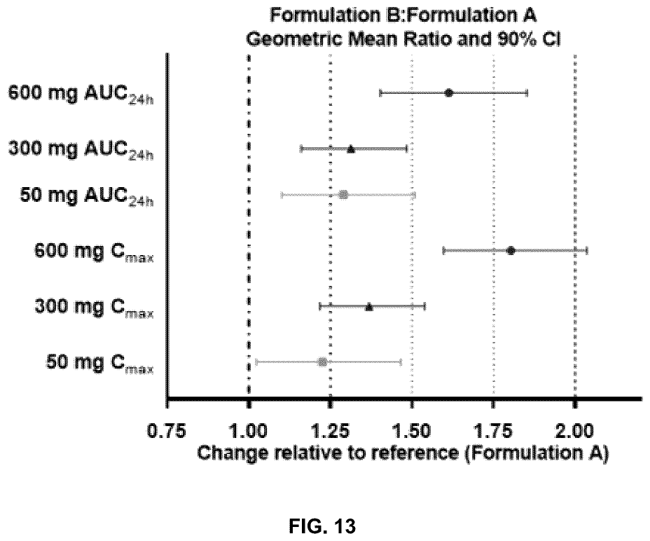

FIG. 13 compares the Geometric Mean and 90% Confidence Intervals of the Cₘₐₓ and Area Under the Curve of Formulations A and B.

A summary of the pharmacokinetic data is provided in Table 12.

TABLE 12
Summary of Single Dose Steady State PK Data.

| | Formu- | Tₘₐₓ | Cₘₐₓ | AUC_{24 h} | AUC_{∞} | t_{1/2} |
|---|---|---|---|---|---|---|
| Dose | lation | (h) | (ng/mL) | (ng · h/mL) | (ng · h/mL) | (h) |
| 50 | A | 8 | 192 | 3,610 | 18,600 | 48.4 |
| | B | 4 | 235 | 4,650 | 22,700 | 53 |
| 300 | A | 8 | 330 | 6,770 | 38,900 | 58.2 |
| | B | 4 | 452 | 8,880 | 48,400 | 59.8 |
| 600 | A | 4 | 351 | 7,260 | 42,600 | 54.1 |
| | B | 4 | 638 | 11,700 | 56,900 | 54.9 |
| 1,000 | A | 12 | 505 | 9,670 | 57,500 | 68.5 |

Bezuclastinib was slowly absorbed after oral administration, reaching median time to maximum concentration 12 to 16 hours post dose. Subsequently, plasma concentrations declined in a monophasic manner, with an elimination phase t_{1/2} of 48.6 to 71.4 hours.

Higher bezuclastinib exposure and faster absorption were observed with Formulation B as compared with Formulation A across the dose range of 50 mg to 600 mg and the difference in exposure between the 2 formulations increased with dose. The geometric mean ratios (GMRs) of Formulation B/Formulation A were 1.23, 1.37, and 1.80 for Cₘₐₓ and 1.09, 1.24, and 1.35 for AUC_{0-∞} at doses of 50 mg, 300 mg, and 600 mg, respectively.

Bezuclastinib Cₘₐₓ was slightly higher while AUC_{0-∞} was comparable after a single dose of 600 mg of Formulation B compared with that of 1000 mg of Formulation A with GMR of 1.26 for Cₘₐₓ and 1.10 for AUC_{0-∞}.

Both formulations across dose levels were well tolerated, did not reveal any clinically significant AEs or SAEs. All AEs were of low grade and reversible.

As shown in this Example, Formulation B delivered more drug product (as measured by maximum plasma concentration and AUC), and it did so in a dose-dependent manner. Similar results in rats and non-human primates for SDD formulations with HPMCAS-H, but unexpectedly not with HPMCAS-L, were already shown in the previous Examples. These results led to the selection of Formulation B for further study in combination treatment.

### Example 8: Evaluation of Formulation B Co-Administered with Sunitinib Malate in Humans

Bezuclastinib Formulation B was co-administered with sunitinib malate in humans with GIST in a randomized, open label, multicenter clinical study. Fourteen (14) patients were admitted with histologically confirmed Gastrointestinal Stromal Tumors (GIST) w/at least 1 measurable lesion per mRECIST v1.1 that were locally advanced, unresectable, or metastatic and documented disease progression on or intolerance to Imatinib. Patients received 600 mg dose of bezuclastinib Formulation B with 37.5 mg dose of sunitinib malate once daily to steady state. A summary of the pharmacokinetic data is provided in Table 13.

TABLE 13
Summary of Single Dose Steady State PK Data.

| | Cₘₐₓ | AUC₂₄ₕ |
|---|---|---|
| Formulation/Dose | (ng/mL) | (ng · h/mL) |
| Formulation B 600 mg | 2,090 | 42,100 |
| sunitinib malate 37.5 mg | | |

Surprisingly, exposure and absorption of 600 mg bezuclastinib Formulation B co-administered with 37.5 mg sunitinib malate to steady state was significantly higher than a single, 600 mg dose of bezuclastinib Formulation B alone. This synergistic effect has not previously been observed with other formulations of bezuclastinib co-administered with sunitinib malate.

### Article of Manufacture

The article of manufacture comprises a container holding the tablet suitable for oral administration of Compound (I) in combination with printed labeling instructions providing a discussion of when a particular dosage form should be administered with food and when it should be taken on an empty stomach. The tablet will be contained in any suitable container capable of holding and dispensing.

The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

### EQUIVALENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A spray-dried solid dispersion comprising:
(a) 3,4-dimethyl-N-(2-phenyl-1H-pyrrolo[2,3-b]pyridin-5-yl)-1H-pyrazole-5-carboxamide (Compound (I)),
(b) a pharmaceutically acceptable polymer, and
wherein the 3,4-dimethyl-N-(2-phenyl-1H-pyrrolo[2,3-b]pyridin-5-yl)-1H-pyrazole-5-carboxamide (Compound (I)) is dispersed in a polymer matrix formed from the pharmaceutically acceptable polymer.

2. The spray-dried dispersion of claim 1, wherein the 3,4-dimethyl-N-(2-phenyl-1H-pyrrolo[2,3-b]pyridin-5-yl)-1H-pyrazole-5-carboxamide (Compound (I)) is in an amorphous form or in a free base form.

3. (canceled)

4. The spray-dried solid dispersion of claim 1, wherein the pharmaceutically acceptable polymer is hydroxypropyl methyl cellulose acetate succinate grade H (HPMCAS-H).

5. The spray-dried solid dispersion of claim 1, wherein the spray-dried solid dispersion comprises at least about 1% by weight to at least about 25% by weight of 3,4-dimethyl-N-(2-phenyl-1H-pyrrolo[2,3-b]pyridin-5-yl)-1H-pyrazole-5-carboxamide (Compound (I)).

6. The spray-dried solid dispersion of claim 1, wherein the spray-dried solid dispersion comprises at least about 75% by weight to at least about 99% by weight of the pharmaceutically acceptable polymer.

7. The spray-dried solid dispersion of claim 1, wherein the weight ratio of the 3,4-dimethyl-N-(2-phenyl-1H-pyrrolo[2,3-b]pyridin-5-yl)-1H-pyrazole-5-carboxamide (Compound (I)) to the pharmaceutically acceptable polymer is from about 1:3 to about 1:99.

8. The spray-dried solid dispersion of claim 1, further comprising a solvent.

9. The spray-dried solid dispersion of claim 8, wherein the solvent is a combination of water and tetrahydrofuran.

10. The spray-dried solid dispersion of claim 9, wherein the volume ratio of water to tetrahydrofuran is from about 1:2 to about 1:99.

11. (canceled)

12. (canceled)

13. A tablet comprising:
3,4-dimethyl-N-(2-phenyl-1H-pyrrolo[2,3-b]pyridin-5-yl)-1H-pyrazole-5-carboxamide (Compound (I)) dispersed in a polymer matrix formed from a pharmaceutically acceptable polymer, and
one or more pharmaceutically acceptable ingredients selected from the group consisting of one or more binders, one or more buffering agents, one or more diluents, one or more disintegrants, one or more fillers, one or more film coatings, one or more lubricants, one or more glidants, and one or more surfactants.

14. The tablet of claim 13, wherein the 3,4-dimethyl-N-(2-phenyl-1H-pyrrolo[2,3-b]pyridine-5-yl)-1H-pyrazole-5-carboxamide (Compound (I)) dispersed in a polymer matrix formed from a pharmaceutically acceptable polymer is a spray-dried solid dispersion.

15. The tablet of claim 13, wherein the 3,4-dimethyl-N-(2-phenyl-1H-pyrrolo[2,3-b]pyridine-5-yl)-1H-pyrazole-5-carboxamide (Compound (I)) is in an amorphous form or in a free base form.

16. (canceled)

17. The tablet of claim 13, wherein the pharmaceutically acceptable polymer is hydroxypropyl methyl cellulose acetate succinate grade H (HPMCAS-H).

18. The tablet of claim 13, wherein the one or more pharmaceutically acceptable ingredients comprise colloidal silicon dioxide, croscarmellose sodium, sodium stearyl fumarate, mannitol, and microcrystalline cellulose.

19. The tablet of claim 13, wherein the tablet comprises at least about 1% by weight to at least about 20% by weight of 3,4-dimethyl-N-(2-phenyl-1H-pyrrolo[2,3-b]pyridin-5-yl)-1H-pyrazole-5-carboxamide (Compound (I)).

20. The tablet of claim 13, wherein the tablet comprises at least about 10% by weight to at least about 90% by weight of the pharmaceutically acceptable polymer.

21. The tablet of claim 13, wherein the tablet comprises at least about 3% by weight to at least about 65% by weight of the one or more pharmaceutically acceptable ingredients selected from the group consisting of one or more binders, one or more buffering agents, one or more diluents, one or more disintegrants, one or more fillers, one or more lubricants, one or more glidants, and one or more surfactants.

22. (canceled)

23. A method of treating a subject suffering from disease or condition selected from Acute Myeloid Leukemia (AML), Gastrointestinal Stromal Tumors (GIST), Mast Cell Leukemia (MCL) and mastocytosis comprising administering orally to the subject the tablet of claim 13.

24. The method of claim 23, further comprising administering to the subject a therapeutic agent in combination with the tablet.

25. The method of claim 24, wherein the therapeutic agent is sunitinib malate.

26. The method of claim 23, wherein the disease or condition is Acute Myeloid Leukemia (AML), Gastrointestinal Stromal Tumors (GIST), or mastocytosis.

27. (canceled)

28. (canceled)

29. The method of claim 26, wherein the mastocytosis is Advanced Systemic Mastocytosis (AdvSM), Nonadvanced Systemic Mastocytosis (NonAdvSM), or Indolent Systemic Mastocytosis (ISM) and Smoldering Systemic Mastocytosis (SSM).

30-33. (canceled)

34. The method of claim 23, wherein the tablet is taken continuously in 28-day cycles.

35. The method of claim 23, wherein single dose target area-under-the-curve (AUC) is from 500 to 80,000 (ng·h/mL).

36. The method of claim 23, wherein single dose maximal plasma concentration (Cₘₐₓ) is from 100 to 800 (ng/mL).

37. The method of claim 25, wherein once daily, steady state target area-under-the-curve (AUC) is from 30,000 to 50,000 (ng·h/mL) when 600 mg dose of bezuclastinib is co-administered with 37.5 mg of sunitinib malate.

38. The method of claim 25, wherein the once daily, steady state maximal plasma concentration (Cₘₐₓ) is from 1,500 to 2,500 (ng/mL) when 600 mg doses of bezuclastinib are co-administered with 37.5 mg of sunitinib malate.
